(19) United States
(12) Patent Application Publication
(10) Pub. No.: US 2003/0087863 A1
(43) Pub. Date: May 8, 2003

(54) Methods for diagnosing and treating a disease mediated by decreased MMP-2 function

(75) Inventors: John A. Martignetti, Chappaqua, NY (US); Robert J. Desnick, New York, NY (US)

Correspondence Address: DARBY & DARBY P.C., Post Office Box 5257, New York, NY, 10150-5257, US

(73) Assignee: Mount Sinai School of Medicine

(21) Appl. No.: 10/185,433

(22) Filed: Jun. 28, 2002

Related U.S. Application Data
(60) Provisional application No. 60/301,694, filed on Jun. 28, 2001.

(51) Int. Cl. A61K 48/00; A61K 38/46; A61K 39/395

(52) U.S. Cl. 514/44; 424/146.1; 424/94.65

(57) ABSTRACT

The present invention relates to a method for the prevention or treatment of a disease mediated by decreased MMP-2 function. This may result from an aberrant interaction of molecules that stimulate or inhibit MMP-2 protein synthesis, stability, or function, as well as from mutations in the coding or regulatory regions of the gene encoding MMP-2. The invention also provides a method for identifying a substance useful in this context. It further contemplates a method for diagnosing such a disease.

This application claims priority under 35 U.S.C. §119(e) of provisional application Serial No. 60/301,694 filed Jun. 28, 2001, which is incorporated herein by reference in its entirety.

### FIELD OF THE INVENTION

The present invention relates to a method for the prevention or treatment of a disease mediated by decreased MMP-2 function. This may result from an aberrant interaction of molecules that stimulate or inhibit MMP-2 protein synthesis, stability, or function, as well as from mutations in the coding or regulatory regions of the gene encoding MMP-2. The invention also provides a method for identifying a substance useful in this context. It further contemplates a method for diagnosing such a disease.

### BACKGROUND OF THE INVENTION

The matrix metalloproteases (a.k.a. matrix metalloendo-proteinases or MMPs) are a family of zinc endoproteinases which include, but are not limited to, interstitial collagenase (MMP-1), stromelysin (proteoglycanase, transin, or MMP-3), gelatinase A (72 kda-gelatinase or MMP-2) and gelatinase B (95 kDa-gelatinase or MMP-9). These MMPs are secreted along with natural proteinaceous inhibitors by a variety of cells including fibroblasts and chondrocytes.

MMPs are known to degrade the extracellular matrix during tissue remodeling and are involved in various critical cellular processes including cell migration, proliferation, and apoptosis (Vu et al., Genes Dev, 14(17), 2123-33 ( 2000)). Previous in vitro studies, based on a number of disease states and pathologic conditions suggested that MMP over-expression and increased activity resulted in bone, cartilage, and joint destruction and abnormal wound repair (Konttinen, Y. T., et al., Matrix Biol,. 17(8-9),585-601(1998); Papet al., Arthritis Rheum 2000; 43(6), 1226-32). Several patents and patent applications have therefore proposed to use inhibitors of matrix metalloproteases to treat various conditions such as osteoarthritis, osteopenias (U.S. Pat. No. 6,225,314) or to reduce hair growth (U.S. Pat. No. 5,962,466).

The MMPs are though to achieve biologic effects by two important pathways. First, they have important functions as mediators of extracellular matrix turnover. While their in vivo substrate preferences have not been fully characterized, in vitro substrates include collagens, fibronectin, vitronectin, aggrecan, and laminin among others. This wide substrate preference ensures their role in many normal developmental and tissue repair processes including morphogenesis, angiogenesis, skelatogenesis, and wound healing, as well as some pathological tissue reshaping processes, such as the arthritic erosion of joints. Second, the MMPs are also thought to process a number of ECM-dependent and independent growth factors, cytokines, and other proteinases. These cellular signals can in turn modulate activities such as cell migration, proliferation, and apoptosis.

Among the MMPs, MMP-2 is involved in the hydrolysis of gelatin and type IV collagen, the major structural components of the basement membrane, as well as elastin, laminin, fibronectin, aggrecan, and fibrillin (Yu, A. E., et al., Matrix Metalloproteinases (eds Parks, WC & Mecham, RP) 85-113 (Academic Press, San Diego, 1999)). MMP-2 was originally isolated from the media of cultured rheumatoid synovial cells (Harris, E. D. Jr, & Krane, S. M., Biochim. Biophys. Acta. 258, 566-576 (1972)) and was thought to be involved in normal collagen turnover (Creemers, L. B. et al., Matrix Biol. 17, 35-46 (1998)) and tumor cell invasiveness (Chen, W. T., Enzyme Protein 49, 59-71 (1996)). MMP-2 is expressed in mesenchymal tissues during embryogenic and regenerative remodeling (Karelina, T. V. et al., J. Invest. Dermatol. 114, 371-5(2000); Kanwar, Y. S. et al., Am. J. Physiol. 277, F934-947 (1999)). MMP-2 is also believed to play a role in the processing and regulation of cytokines involved in inflammation, including TNF-α, TGF-β2, IL-1β, and MCP-3.

Paradoxically, the present invention is based on the surprising discovery that in vivo MMP-2 deficiency or inactivity causes bone and joint pathophysiology, abnormal wound healing, as well as hirsutism. These in vivo data are unexpected and counter-intuitive and introduce new perspectives in the diagnosis and treatment of various diseases wherein MMP-2 deficiency or inactivity is observed, including abnormal extracellular matrix metabolism and downstream signaling defects.

### SUMMARY OF THE INVENTION

The present invention relates to a method for the prevention or treatment of a disease mediated by a decreased MMP-2 function, which method comprises stimulating MMP-2 production or activity in the subject.

In one embodiment, this method comprises administering to the subject in need of such treatment an effective amount of a substance that stimulates MMP-2 activity, with a pharmaceutically acceptable carrier. In a specific embodiment, the substance is the MMP-2 activator, MT1-MMP. Alternatively, stimulating MMP-2 activity involves inhibiting the activity of TIMP-2, e.g., with an anti-TIMP-2 antibody, small molecule inhibitor of TIMP-2 modulation of MMP-2, or by inhibiting expression of TIMP-2.

In another embodiment, the method comprises administering to the subject in need of such treatment an effective amount of a vector that encodes an MMP-2 protein, with a pharmaceutically acceptable carrier. This vector may be a DNA vector.

In another embodiment, the method of the invention comprises administering to the subject in need of such treatment an effective amount of an MMP-2 protein, with a pharmaceutically acceptable carrier, i.e., an enzyme replacement therapy regimen.

The disease or disorder may be a syndrome such as Multicentric Osteolysis with Nodulosis and Arthritis (MONA), or may involve arthritis, osteolysis, osteopenia or osteoporosis, hirsutism, abnormal wound healing, keloids, or a desmoid tumor.

The present invention also provides methods for administration of the compositions. In one embodiment, the preferred route of administration is topical.

A further subject of the present invention is a pharmaceutical composition comprising a nucleic acid that encodes an MMP-2 protein, with a pharmaceutically acceptable carrier.

Another subject of the invention is a pharmaceutical composition comprising an MMP-2 protein, with a pharmaceutically acceptable carrier.

Still other pharmaceutical compositions can comprise an MMP-2 activator, e.g., MT1-MMP, or an inhibitor of TIMP-2, such as an anti-TIMP-2 antibody.

The present invention also provides a method for identifying a substance useful in the prevention or treatment of a disease mediated by decreased MMP-2 function, which method comprises determining the effect of the substance on a biological activity of MMP-2 protein, wherein a stimulatory effect is indicative of a substance useful in the prevention or treatment of a disease mediated a deficiency in MMP-2 activity.

In one embodiment, determining the effect of the substance on a biological activity of MMP-2 protein encompasses determining whether the substance has an agonist effect toward binding of MT1-MMP to MMP-2, whereby MMP-2 is activated.

Alternatively determining the effect of the substance on a biological activity of MMP-2 protein encompasses determining whether the substance has an antagonist effect toward binding of TIMP-2-to MMP-2, whereby MMP-2 inhibition is blocked.

The present invention further contemplates a method for diagnosing a disease mediated by a decreased MMP-2 function, which method comprises assessing the level of activity or expression of MMP-2 in a biological sample of a test subject and comparing it to the level of activity or expression of MMP-2 in a control sample, wherein a decrease of activity or expression of MMP-2 in the sample of the test subject compared to the control sample is indicative of such a disease. Here again the disease or disorder may be a MONA syndrome, or may involve arthritis, osteolysis, osteopenia or osteoporosis, hirsutism, abnormal wound healing, keloids, or a desmoid tumor.

In another embodiment, the present invention involves diagnosing, screening or monitoring for diseases by determining MMP-2 mutations in subjects. In a preferred embodiment, the present invention involves determining the presence or absence of specific mutations in MMP-2 genes in subjects with bone diseases.

In this diagnostic method the level of expression of MMP-2 may be assessed by determining the quantity of MMP-2 protein present in the biological sample. It also may be assessed by assaying the quantity of mRNA which is present in the biological sample and encodes MMP-2.

A further subject of the present invention is a method for the prevention or treatment of baldness or alopecia in a subject, which method comprises administering to the subject in need of such treatment an effective amount of a substance that inhibits MMP-2 activity, with a pharmaceutically acceptable carrier.

A further subject of the present invention is a method for removal of hair in a subject, which method comprises stimulating MMP-2 activity in the subject. This method may comprise administering to the subject in need of such treatment an effective amount of a substance that stimulates MMP-2 activity.

### BRIEF DESCRIPTION OF THE DRAWINGS

FIG. 1 shows pedigrees and haplotypes of the Saudi kindreds. Family 1 is believed to be related based on common surname and shared haplotype within the disease gene locus. Affected individuals and disease haplotypes are indicated. A number of markers were found to undergo mutation, most likely secondary to strand slippage, and gave rise to new alleles. These alleles are underlined.

FIGS. 2A-B show gelatin zymography of control and affected serum samples. Lane 1, MMP-2 and MMP-9 zymography standards (Chemicon International, CA). Lane 2 represents serum from an unaffected, unrelated individual, and lanes 3, 4, and 5, are sera from the unaffected parents and sibling in Family 1. Lanes 6 and 7 represent sera from affected children from another arm of Family 1; lanes 8 and 9, sera from affected children from Family 3; and lane 10, serum from the affected child in Family 2. B. Gelatin zymography of control and patient fibroblast conditioned media. Lane 1 represents a mixture of MMP-2 and MMP-9 zymogram standards; lane 2, serum from unrelated, unaffected individual; and lanes 3 and 4, sera from two affected members of Family 3.

FIG. 3 is a schematic drawing of the organization of the MMP-2 gene. The MMP-2 gene has 13 exons of which exons 1 through 4 and 8 through 12 show extensive homology to the interstitial collagenase and stromelysin genes while exons 5 through 7 each encode one complete internal repeat, resembling the collagen-binding domains of the fibronectin type II (Huhtala, P. et al., Genomics 6, 554-559 (1990)).

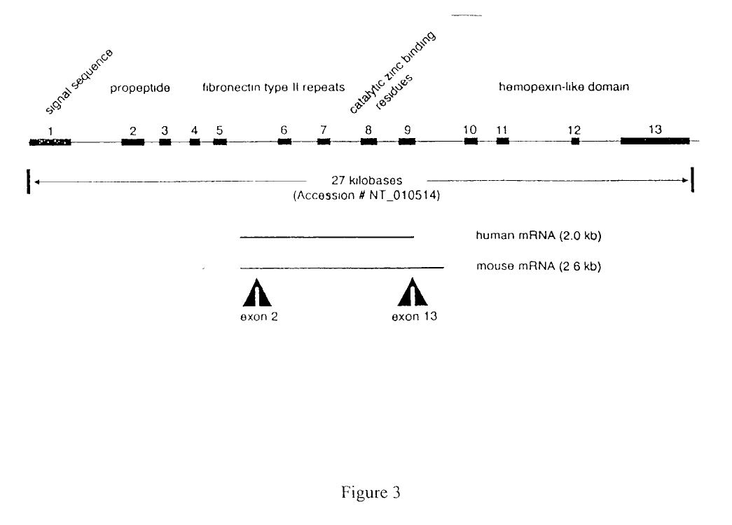

FIGS. 4A-D shows results of DEXA (dual energy X-ray absorptometry) studies performed using age and litter-matched mice. Marked bone density losses of approximately 10-20% were present in femurs and spine from hypomorphic mice (4A and 4D) when compared to control littermates.

FIGS. 5A-D show whole body X-ray imaging of homozygous MMP-2 deficient mice and control littermates. X-rays suggested a time-dependent loss of bone mineral density in homozygous MMP-2 deficient mice (6B and 6D) compared with wild-type controls (6A and 6C).

FIGS. 6A-B show results of immunostaining of Mouse bone marrow cells isolated from paired homozygous MMP-2 deficient and wild-type mice and plated in the presence of ascorbate and washed after 36 hours to remove non-adherent cells. The wild-type cells formed colonies (6A) and large mineralized clusters of osteoblasts, as indicated by staining with alkaline phosphatase (AP). MMP-2 deficient colonies (6B) were sparse and low in cell number.

FIG. 7 is a histogram of MMP-2 activity (ng/mL) in serum from control and arthritic patients. The Y-axis represents activity of MMP-2, and X-axis represents patient identification numbers. Patient numbers 1-3 represent control group; numbers 4-8 represent psoriatics; numbers 9-17 are patients negative for rheumatoid factor; numbers 18-36 are from patients with increasing amounts of rheumatoid factor; sample number 37 represents a positive control; and patient numbers 38 and 39 represent non-MONA conditions.

FIG. 8 represents a histogram of MMP-2 protein levels in serum from control and arthritic patients. The Y-axis represents activity of MMP-2 concentration (ng/mg protein), and X-axis represents patient identification numbers. Patient numbers 1-10 represent control group who are rheumatoid factor negative; numbers 11-15 represent psoriatics; number 16 represents a positive control for MMP-2; and numbers 17-35 are from patients with increasing amount of rheumatoid factor.

### DETAILED DESCRIPTION

The present invention is based, in part, on the discovery that a MMP gene is mutated in an inherited osteolysis condition. This established, for the first time, that certain diseases and disorders could result from decreased levels of MMP-2 activity. Previously, increased MMP-2 activity was associated with disease conditions. The present invention advantageously provides methods for preventing or treating a disease mediated by a deficiency of MMP-2 activity, whether such a deficiency occurs as a result of (i) a mutation in the regulatory region or coding region of one or both alleles for MMP-2 that results in a reduction in the level of expression or elimination of expression of MMP-2; (ii) a mutation in the regulatory region or coding region of one or both alleles for MMP-2 that results in expression of a defective MMP-2 protein, whether or not the absolute amount of such protein remains at normal levels; (iii) a deficiency of MT1-MMP activity, resulting in insufficient activation of normal levels of endogenous MMP-2; and (iv) over-activity of an MMP-2 inhibitor such as TIMP-2, resulting in suppression of normal levels of endogenous MMP-2. These various causes of decreased MMP-2 activity can manifest as an extracellular matrix breakdown defect or a defect in downstream signaling mediated by MMP-2. The invention further provides for determining the presence of such a disease (diagnosis), the likelihood of developing such a disease (predisposition), or the status and expected course of such a disease (prognosis) based on detecting a reduction of MMP-2 activity due to any of the foregoing reasons.

The multicentric osteolyses or “vanishing bone” syndromes are a group of autosomal dominant andrecessive skeletal disorders of unknown etiology characterized by progressive bone loss and joint destruction (Hardegger et al., J Bone Joint Surg Br, 67(1):88-93, 1985; Pai et al. Am J Med Genet, 29(4):929-36, 1988; Petit et al., Am J Med Genet, 25(3):537-41, 1986; Szoke, G., et al., Clin Orthop, (310):120-9., 1995; Torg, J. S., et al., J Pediatr, 75(2): 243-52, 1969; Torg, J. S et al., J Bone Joint Surg Am, 50(8):1629-38, 1968; Urlus, M., et al., Genet Couns, 4(1):25-36, 1993).

Investigators have recently identified a new autosomal recessive member of this group, “Multicentric Osteolysis with Nodulosis and Arthritis” (MONA; OMIM #605156), (A1 Aqeel et al., Am J Med Genet 93(1);11-8, 2000). This syndrome is characterized by carpal and tarsal osteolysis, global osteoporosis, arthritic changes, facial dysmorphia, abnormal wound healing, and the development of desmoid tumor-like fibrocollagenous pads.

Using a positional cloning strategy, it has now been discovered that the disease is caused by mutations in the matrix metalloproteinase 2 gene, the first identified MMP deficiency. The present invention, thus, advantageously establishes a role for MMP-2 deficiency in the development of MONA pathology. This work provides the basis for associating an in vivo MMP-2 deficiency to various diseases (i.e., diseases, disorders, conditions, syndromes etc.) that show the same symptoms as the MONA syndrome. Such diseases include arthritis, osteolysis, osteopenia, hirsutism, and abnormal wound healing. Keloids and desmoid tumors resulting from such extracellular matrix breakdown defects are also encompassed. More generally, all these diseases are herein referred to as diseases mediated by decreased MMP-2 function.

Furthermore, while MMP-2 deficiency in humans results in MONA syndrome, MMP-2 deficient mice have been described as being overtly normal (Itoh et al, J. Biol Chem, 1997 272(36): 22389-92). However, they are approximately 15% smaller than control littermates and this mild, but obvious, phenotype which may be secondary to a skeletal defect has not been investigated. The present invention provides an understanding of this otherwise unnoticeable growth defect, and an explanation for the otherwise overtly normal phenotypes of these mice: These mice are not true knockouts but possess low levels of active enzyme. This low level of MMP-2 activity may explain the difference between the human (MONA) and murine (mild growth restriction) phenotypes. This suggests that the marked decrease in mouse growth is secondary to a skeletal defect and thus these mice provide a critical investigational tool for understanding the role of MMP-2 in skeletal growth. These mice further confirm that heterozygous individuals, or individuals with inactivation of MMP-2, should be identified to better understand and characterize any defects or disorders.

These results further provide a basis for investigating whether subjects with a short stature, including subjects suffering from dwarfism, show a deficiency or an inactivation of MMP-2. Consequently the present invention also provides methods and compositions, which may be used, e.g., for enhancing the growth of the subjects that show a deficiency of MMP-2, by stimulating their MMP-2 activity.

### MMP-2 Activity or Function

An “MMP-2 activity” or “MMP-2 biological activity” (“MMP-2 function”) refers to functional property shown by the wild-type MMP-2 protein in vivo or in vitro. This may include a collagenase activity, that may be assayed by zymography, collagen lattice assays or in vitro collagen dissolution assays (Havemose-Poulsen et al, J. Periodontal Research, 1998, vol 33:280-291). Other examples of MMP-2 activity include the interaction of MMP-2 protein to other molecules such as MT1-MMP, TIMP-2, integrin α5β3, MCP-3 protein, or other physiologically relevant substrate, activator or receptor.

As used herein, the term “MMP-2 deficiency” refers to both deficient quantities of MMP-2 protein and reduced or abrogated MMP-2 protein activity (e.g., due to an inactivating mutation in a binding or activation domain, insufficient activity of an endogenous activator like MT1-MMP, or over-activity of an MMP-2 inhibitor like TIMP-2). Thus, a reduction in MMP-2 activity can result from the presence of less protein, or the presence of a normal amount of protein having lower activity as a result of a mutation or because of deregulation of its activity. Such MMP-2 deficiencies result in decreased MMP-2 function.

As used herein the term “MMP-2 protein” refers to the matrix metalloproteinase 2, also known as gelatinase A, collagenase type IV, or EC3.4.24.24. The terms “polypeptide” and “protein” may be used interchangeably to refer to the gene product (or corresponding synthetic product) of a MMP-2 gene. The term “protein” also may refer specifically to the polypeptide as expressed in cells.

This term encompasses the MMP-2 protein of human origin, that has an amino acid sequence available on Swissprot database (access number #P08253). It also encompasses function-conservative variants and homologous proteins thereof, proteins originating from different species.

As used herein the term “MMP-2 nucleic acid” refers to a polynucleotide that encodes an MMP-2 protein as described above.

An “MMP-2 gene” is used herein to refer to a portion of a DNA molecule that includes an MMP-2 polypeptide coding sequence operatively associated with expression control sequences. Thus, a gene includes both transcribed and untranscribed regions. The transcribed region may include introns, which are spliced out of the mRNA, and 5′- and 3′-untranslated (UTR) sequences along with protein coding sequences. In some embodiments, the gene can be a genomic or partial genomic sequence, in that it contains one or more introns. In other embodiments, the term gene may refer to a cDNA molecule (i.e., the coding sequence lacking introns).

The terms “MMP-2 gene” or “MMP-2 nucleic acid” more particularly encompass sequence-conservative variants as well as homologous sequences, such as allelic variants of (or) species variants.

“Sequence-conservative variants” of a polynucleotide sequence are those in which a change of one or more nucleotides in a given codon position results in no alteration in the amino acid encoded at that position.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, most preferably at least 85%, and even more preferably at least 90%, and which has the same or substantially similar properties or functions as the native or parent protein or enzyme to which it is compared.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell 50:667, 1987). Such proteins (and their encoding genes) have sequence homology, as reflected by their sequence similarity, whether in terms of percent similarity or the presence of specific residues or motifs at conserved positions.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In a specific embodiment, two DNA sequences are “substantially homologous” or “substantially similar” when at least about 80%, and most preferably at least about 90 or 95% of the nucleotides match over the defined length of the DNA sequences, as determined by sequence comparison algorithms, such as BLAST, FASTA, DNA Strider, etc. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially similar” when greater than 80% of the amino acids are identical, or greater than about 90% are similar (functionally identical). Preferably, the similar or homologous sequences are identified by alignment using, for example, the GCG (Genetics Computer Group, Program Manual for the GCG Package, Version 7, Madison, Wis.) pileup program, or any of the programs described above (BLAST, FASTA, etc.).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., infra). The conditions of temperature and ionic strength determine the “stringency” of the hybridization. For preliminary screening for homologous nucleic acids, low stringency hybridization conditions, corresponding to a Tm (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. SSC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementarity, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., infra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

### Therapeutic Applications

The present invention contemplates that the stimulation of MMP-2 activity in a subject is useful in the prevention or treatment of a disease mediated by decreased MMP-2 function.

A “subject” is a human or an animal likely to develop such disease, more particularly a mammal, such as a rodent or a primate. Preferably the subject is a human, particularly as the natural condition resulting from an MMP-2 deficiency is a human disease.

The term “prevention” refers to the prevention of the onset of the disease, which means to prophylactically interfere with a pathological mechanism that results in the disease or disorder. In the context of the present invention, such a pathological mechanism can be a decrease in MMP-2 expression or activity. The patient may be a subject that has an increased risk of developing the disease.

The term “treatment” means to therapeutically intervene in the development of a disease in a subject showing a symptom of this disease. In the context of the present invention, these symptoms can include arthritis, osteolysis, osteopenia or osteoporosis, hirsutism, abnormal wound healing, and keloids, or a desmoid tumor.

The term “therapeutically effective amount” is used herein to mean an amount or dose sufficient to augment the level of MMP-2 activity e.g., by about 10 percent, preferably by about 50 percent, and more preferably by about 90 percent. Preferably, a therapeutically effective amount can ameliorate or prevent a clinically significant deficit in the activity, function and response of the subject. Alternatively, a therapeutically effective amount is sufficient to cause an improvement in a clinically significant condition in the subject.

As used herein, the term “stimulating MMP-2 activity” means either enhancing the MMP-2 activity observed in a subject or generating an MMP-2 activity in a subject that shows an absence or deficiency of such activity.

MMP-2 activity can be stimulated by various methods, including delivery of a gene therapy vector that produces MMP-2; enzyme replacement therapy with an MMP-2 protein; activation of endogenous MMP-2 through increasing the activity of an MMP-2 activator like TM1-MMP (which can be achieved through gene therapy or by administering TM1 protein); or activation of endogenous MMP-2 through suppression of an MMP-2 inhibitor like TIMP-2 (which can be achieved through gene therapy to suppress protein expression, e.g., with antisense technology, or by administering an inhibitor like an anti-TIMP-2 antibody); as described hereafter.

### Gene Therapy

In one embodiment of the present invention, the method for the prevention or treatment of a disease mediated by decreased MMP-2 function, comprises administering to the subject in need of such treatment an effective amount of a vector that encodes an MMP-2 protein, with a pharmaceutically acceptable carrier.

The term “vector” means the vehicle by which a DNA or RNA sequence (e.g. a foreign gene) can be introduced into a host cell, so as to transform the host and promote expression (e.g. transcription and translation) of the introduced sequence. Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. In the context of the present invention, the vector that encodes an MMP-2 protein is a vehicle by which a nucleic acid that encodes an MMP-2 protein in association with expression control sequences is introduced into a host cell.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

A coding sequence is “under the control of” or “operatively associated with” transcriptional and translational control sequences in a cell when RNA polymerase transcribes the coding sequence into RNA, particularly mRNA, which is then trans-RNA spliced (if it contains introns) and translated into the protein encoded by the coding sequence.

The “expression control sequences” are transcriptional or translational control sequences including enhancer, repressor or promoter sequences.

A “promoter” or “promoter sequence” is a DNA regulatory region capable of binding RNA polymerase in a cell and initiating transcription of a downstream (3′ direction) coding sequence. For purposes of defining the present invention, the promoter sequence is bound at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme.

The term “expression system” means a host cell and compatible vector under suitable conditions, e.g. for the expression of a protein coded for by foreign DNA carried by the vector and introduced to the host cell. Expression systems particularly useful in gene therapy are discussed in greater detail below.

### Expression Systems

A wide variety of host/expression vector combinations (i.e., expression systems) may be employed in expressing MMP-2. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX (Smith et al., Gene 67:31-40, 1988), pMB9 and their derivatives, plasmids such as RP4; gram positive vectors such as Strep. gardonii; phage DNAS, e.g., the numerous derivatives of phage 1, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Expression of the protein or polypeptide may be controlled by any promoter/enhancer element known in the art, but these regulatory elements must be functional in the host selected for expression. Promoters which may be used to control gene expression include, but are not limited to, cytomegalovirus (CMV) promoter, the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 1982); prokaryotic expression vectors such as the β-lactamase promoter (Villa-Komaroff, et al, Proc. Natl. Acad. Sci. U.S.A. 75:3727-3731, 1978), or the tac promoter (DeBoer, et al., Proc. Natl. Acad. Sci. U.S.A. 80:21-25, 1983); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and control regions that exhibit hematopoietic tissue specificity, in particular: beta-globin gene control region which is active in myeloid cells (Mogram et al, Nature 315:338-340, 1985; Kollias et al., Cell 46:89-94, 1986), hematopoietic stem cell differentiation factor promoters, erythropoietin receptor promoter (Maouche et al., Blood, 15:2557, 1991), etc; and control regions that exhibit mucosal epithelial cell specificity.

Preferred vectors are viral vectors, such as lentiviruses, retroviruses, herpes viruses, adenoviruses, adeno-associated viruses, vaccinia viruses, baculoviruses, alpha viruses and other recombinant viruses with desirable cellular tropism. Thus, a vector encoding an MMP-2 protein can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published Oct. 1995.

Viral vectors commonly used for in vivo or ex vivo targeting and vaccination procedures are DNA-based vectors and retroviral vectors. Methods for constructing and using viral vectors are known in the art (see, e.g., Miller and Rosman, BioTechniques, 7:980-990, 1992). Preferably, the viral vectors are replication defective, that is, they are unable to replicate autonomously in the target cell. Preferably, the replication defective virus is a minimal virus, i.e., it retains only the sequences of its genome which are necessary for encapsidating the genome to produce viral particles.

DNA viral vectors include an attenuated or defective DNA virus, such as but not limited to herpes simplex virus (HSV), papillomavirus, Epstein Barr virus (EBV), adenovirus, adeno-associated virus (AAV), vaccinia virus, Venezuelan Equine Encephalitis Virus (VEEV), and the like. Examples of particular vectors include, but are not limited to, a defective herpes virus 1 (HSV1) vector (Kaplitt et al., Molec. Cell. Neurosci. 2:320-330, 1991; International Patent Publication No. WO 94/21807, published Sep. 29, 1994; International Patent Publication No. WO 92/05263, published Apr. 2, 1994); an attenuated adenovirus vector, such as the vector described by Stratford-Perricaudet et al. (J. Clin. Invest. 90:626-630, 1992; see also La Salle et al., Science 259:988-990, 1993); and a defective adeno-associated virus vector (Samulski et al., J. Virol. 61:3096-3101, 1987; Samulski et al., J. Virol. 63:3822-3828, 1989; Lebkowski et al., Mol. Cell. Biol. 8:3988-3996, 1988).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Adenovirus vectors. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5) or adenoviruses of animal origin (see WO94/26914). Those adenoviruses of animal origin which can be used within the scope of the present invention include adenoviruses of canine, bovine, murine (for example: Mavl, Beard et al., Virology 75 (1990) 81), ovine, porcine, avian, and simian (example: SAV) origin. Preferably, the adenovirus of animal origin is a canine adenovirus, more preferably a CAV2 adenovirus (e.g. Manhattan or A26/61 strain (ATCC VR-800), for example). Various replication defective adenovirus and minimum adenovirus vectors have been described (WO94/26914, WO95/02697, WO94/28938, WO94/28152, WO94/12649, WO95/02697 WO96/22378). The replication defective recombinant adenoviruses according to the invention can be prepared by any technique known to the person skilled in the art (Levrero et al., Gene 101:195 1991; EP 185 573; Graham, EMBO J. 3:2917, 1984; Graham et al., J. Gen. Virol. 36:59, 1977). Recombinant adenoviruses are recovered and purified using standard molecular biological techniques, which are well known to one of ordinary skill in the art.

Adeno-associated viruses. The adeno-associated viruses (AAV) are DNA viruses of relatively small size which can integrate, in a stable and site-specific manner, into the genome of the cells which they infect. They are able to infect a wide spectrum of cells without inducing effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see WO 91/18088; WO 93/09239; U.S. Pat. Nos. 4,797,368, 5,139,941, EP 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants which are produced are then purified by standard techniques.

Retroviris vectors. In another embodiment the gene can be introduced in a retroviral vector, e.g., as described in Anderson et al., U.S. Pat. No. 5,399,346; Mann et al., Cell 33:153 1983, Temin et al, U.S. Pat. No. 4,650,764; Temin et al., U.S. Pat. No. 4,980,289; Markowitz et al., J. Virol. 62:1120 1988, Temin et al., U.S. Pat. No. 5,124,263; EP 453242, EP178220; Bernstein et al. Genet. Eng. 7 (1985) 235; McCormick, BioTechnology 3 (1985) 689; International Patent Publication No. WO 95/07358, published Mar. 16, 1995, by Dougherty et al.; and Kuo et al., Blood 82:845, 1993,. The retroviruses are integrating viruses which infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PA317 (U.S. Pat. No. 4,861,719); the PsiCRIP cell line (WO 90/02806) and the GP+envAm-12 cell line (WO 89/07150). In addition, the recombinant retroviral vectors can contain modifications within the LTRs for suppressing transcriptional activity as well as extensive encapsidation sequences which may include a part of the gag gene (Bender et al., J. Virol. 61:1639, 1987). Recombinant retroviral vectors are purified by standard techniques known to those having ordinary skill in the art.

Retrovirus vectors can also be introduced by DNA viruses, which permits one cycle of retroviral replication and amplifies tranfection efficiency (see WO 95/22617, WO 95/26411, WO 96/39036, WO 97/19182).

Lentivirus vectors. In another embodiment, lentiviral vectors can be used as agents for the direct delivery and sustained expression of a transgene in several tissue types, including brain, retina, muscle, liver and blood. The vectors can efficiently transduce dividing and nondividing cells in these tissues, and maintain long-term expression of the gene of interest. For a review, see, Naldini, Curr. Opin. Biotechnol., 9:457-63, 1998; see also Zufferey, et al., J. Virol., 72:9873-80, 1998). Lentiviral packaging cell lines are available and known generally in the art. They facilitate the production of high-titer lentivirus vectors for gene therapy. An example is a tetracycline-inducible VSV-G pseudotyped lentivirus packaging cell line which can generate virusparticles at titers greater than 106 IU/ml for at least 3 to 4 days (Kafri, et al., J. Virol, 73: 576-584, 1999). The vector produced by the inducible cell line can be concentrated as needed for efficiently transducing nondividing cells in vitro and in vivo.

Non-viral vectors. In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner, et. al., Proc. Natl. Acad. Sci. U.S.A. 84:7413-7417, 1987; Felgner and Ringold, Science 337:387-388, 1989; see Mackey, et al., Proc. Natl. Acad. Sci. U.S.A. 85:8027-8031, 1988; Ulmer et al., Science 259:1745-1748, 1993). Useful lipid compounds and compositions for transfer of nucleic acids are described in International Patent Publications WO95/18863 and WO96/17823, and in U.S. Pat. No. 5,459,127. Lipids may be chemically coupled to other molecules for the purpose of targeting (see Mackey, et al., supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., International Patent Publication WO95/21931), peptides derived from DNA binding proteins (e.g., International Patent Publication WO96/25508), or a cationic polymer (e.g., International Patent Publication WO95/21931).

It is also possible to introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun (ballistic transfection), or use of a DNA vector transporter (see, e.g., Wu et al., J. Biol. Chem. 267:963-967, 1992; Wu and Wu, J. Biol. Chem. 263:14621-14624, 1988; Hartmut et al., Canadian Patent Application No. 2,012,311, filed Mar. 15, 1990; Williams et al., Proc. Natl. Acad. Sci. USA 88:2726-2730, 1991). Receptor-mediated DNA delivery approaches can also be used (Curiel et al., Hum. Gene Ther. 3:147-154, 1992; Wu and Wu, J. Biol. Chem. 262:4429-4432, 1987). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C. P. Acad. Sci., 321:893, 1998; WO 99/01157; WO 99/01158; WO 99/01175).

Methods for administering such vectors to a subject in need of such treatment are further described hereafter.

Any of the methods for gene therapy available in the art can be used according to the present invention. Exemplary methods are described below. For general reviews of the methods of gene therapy, see, Goldspiel et al., Clinical Pharmacy 1993, 12:488-505; Wu and Wu, Biotherapy 1991, 3:87-95; Tolstoshev, Ann. Rev. Pharmacol. Toxicol. 1993, 32:573-596; Mulligan, Science 1993, 260:926-932; and Morgan and Anderson, Ann. Rev. Biochem. 1993, 62:191-217; May, TIBTECH 1993, 11:155-215. Methods commonly known in the art of recombinant DNA technology that can be used are described in Ausubel et al., (eds.), 1993, Current Protocols in Molecular Biology, John Wiley & Sons, NY; Kriegler, 1990, Gene Transfer and Expression, A Laboratory Manual, Stockton Press, N.Y.; and in Chapters 12 and 13, Dracopoli et al., (eds.), 1994, Current Protocols in Human Genetics, John Wiley & Sons, NY.

In one embodiment, a vector is used in which the coding sequences and any other desired sequences are flanked by regions that promote homologous recombination at a desired site in the genome, thus providing for expression of the construct from a nucleic acid molecule that has integrated into the genome (Koller and Smithies, Proc. Natl. Acad. Sci. USA 1989, 86:8932-8935; Zijlstra et al, Nature 1989, 342:435-438).

Delivery of the vector into a patient may be either direct, in which case the patient is directly exposed to the vector or a delivery complex, or indirect, in which case, cells are first transformed with the vector in vitro, then transplanted into the patient. These two approaches are known, respectively, as in vivo and ex vivo gene therapy.

In a specific embodiment, the vector is directly administered in vivo, where it enters the cells of the organism and mediates expression of the construct. This can be accomplished by any of numerous methods known in the art and discussed above, e.g., by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont); or coating with lipids or cell-surface receptors or transfecting agents, encapsulation in biopolymers (e.g., poly-β-1-→-4-N-acetylglucosamine polysaccharide; see, U.S. Pat. No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (see, e.g., Wu and Wu, J. Biol. Chem. 1987, 62:4429-4432), etc. In another embodiment, a nucleic acid-ligand complex can be formed in which the ligand comprises a fusogenic viral peptide to disrupt endosomes, allowing the nucleic acid to avoid lysosomal degradation, or cationic 12-mer peptides, e.g., derived from antennapedia, that can be used to transfer therapeutic DNA into cells (Mi et al., Mol. Therapy 2000, 2:339-47). In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188). Additional targeting and delivery methodologies are contemplated in the description of the vectors, below.

Preferably, for in vivo administration of viral vectors, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-γ (IFN-65 ), or anti-CD4 antibody, can be administered to block humoral or cellular immune responses to the viral vectors (see, e.g., Wilson, Nature Medicine, 1995). In that regard, it is advantageous to employ a viral vector that is engineered to express a minimal number of antigens.

### Administration of an MMP-2 Protein

In another embodiment of the present invention, the method for the prevention or treatment of a disease mediated by decreased MMP-2 function comprises administering to the subject in need of such treatment an effective amount of an MMP-2 protein, with a pharmaceutically acceptable carrier. The MMP-2 protein is commercially available, and for example it may be purchased from Chemicon (reference CC071). Alternatively the protein can be conceivably prepared using well-known techniques in peptide synthesis, including solid phase synthesis (using, e.g., BOC of FMOC chemistry), or peptide condensation techniques. It may also be produced in a recombinant system, by culturing a host cell transfected with an expression vector under conditions that result in expression of a nucleic acid codings for an MMP-2 protein according to standard techniques well-known in the art, such as the ones described supra. Preferred expression systems are described in the Examples below.

The polypeptide that is so produced may be recovered and preferably purified. Methods for purification are well-known in the art. The purification methods including, without limitation, preparative disc-gel electrophoresis and isoelectric focusing; affinity, HPLC, reversed-phase HPLC, gel filtration or size exclusion, ion exchange and partition chromatography; precipitation and salting-out chromatography; extraction; and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell. As used herein, the term “substantially free” is used operationally, in the context of analytical testing of the material. Preferably, purified material substantially free of contaminants is at least 50% pure. Purity can be evaluated by chromatography, gel electrophoresis, immunoassay, composition analysis, biological assay, and other methods known in the art.

The present invention also encompasses the administration of a non-peptide compound that mimics the structure of the MMP-2 protein. These compounds are called non-peptide analogs.

The present invention further contemplates the administration of deletion mutant MMP-2 proteins, or fragments of MMP-2 protein, that comprise active domains of the protein.

These active domains include the catalytic domain, the hemopexin domain, the MT1-MMP binding domain, the TIMP-2 binding domain, and the integrin binding domain.

### Identification and Adiniistration of a Substance that Stimulates MMP-2 Activity

In an embodiment of the present invention, the method for the prevention or treatment of a disease mediated by decreased MMP-2 function comprises administering to the subject in need of such treatment an effective amount of a substance that stimulates MMP-2 activity, with a pharmaceutically acceptable carrier.

This substance may be a natural protein that upregulates MMP-2, like MT1-MMP, or it may be any substance readily identified by a screening test.

This screening test encompasses determining the effect of a test substance or a biological activity of MMP-2 protein, wherein a stimulatory effect is indicative of a substance useful in the prevention or treatment of a disease mediated by decreased MMP-2 function.

A “test substance” is a chemically defined compound or mixture of compounds (as in the case of a natural extract or tissue culture supernatant), whose ability to stimulate MMP-2 activity may be defined by various assays. A “test substance” is also referred to as a “candidate drug” in the present description.

In one embodiment, the screening method of the invention comprises determining whether the substance has an agonist effect toward binding of MT1-MMP to MMP-2, whereby MMP-2 is activated.

In another embodiment, the screening method of the invention comprises determining whether the substance has an antagonist effect toward binding of TIMP-2 to MMP-2, whereby MMP-2 inhibition is blocked.

Other various screening methods are described below.

Screening and Chemistry

According to the present invention, the structure of MMP-2 protein in its active form or in its mutant form is useful to identify drugs that are effective in preventing or treating a disease mediated by decreased MMP-2 function.

Rational Drug Design. The invention more particularly defines a method of identifying novel drugs that stimulate MMP-2 activity by using rational drug design methods. Such drugs may be designed so that they mimic an active domain of the MMP-2 protein. Alternatively drugs that interact with an active domain of the MMP-2 protein and activate this domain are advantageous too.

Certain of these domains can be defined by computer molecular modeling methods based on a crystal or other three dimensional structure of the MMP-2 protein and mutants thereof.

These active domains include the catalytic domain, the hemopexin domain, the MT1-MMP binding domain, the TIMP-2 binding domain, and the integrin binding domain, as described above.

The present invention contemplates evaluating potential drug for covalent and non-covalent interactions between MMP-2 and the drug. Computer modeling methods that may be used to evaluate these interactions include, but are not limited to, SYBYL and Monte Carlo computer programs. The present invention contemplates computer algorithms that evaluate bonded and non-bonded interactions. Bonded interactions that may be evaluated include, but are not limited to, bond stretching, rotational strain, and torsional strain. Non-bonded interactions that may be evaluated include van Der Waals forces, hydrogen bonds and dipole-dipole interaction.

The invention provides for development of screening assays, particularly for high throughput screening of molecules that upregulate the activity of MMP-2, e.g., by permitting expression of MMP-2 in quantities greater than can be isolated from natural sources, or in indicator cells that are specially engineered to indicate the activity of MMP-2 expressed after transfection or transformation of the cells.

Any screening technique known in the art can be used to screen for MMP-2 agonists or antagonists. The present invention contemplates screens for small molecule ligands or ligand analogs and mimics, as well as screens for natural ligands that bind to and agonize or antagonize MMP-2 expression activity in vivo. For example, natural products libraries can be screened using assays of the invention for molecules that agonize or antagonize MMP-2 expression or activity.

Another approach uses recombinant bacteriophage to produce large libraries. Using the “phage method” (Scott and Smith, Science 249:386-390, 1990; Cwirla, et al., Proc. Natl. Acad. Sci., 87:6378-6382, 1990; Devlin et al., Science, 49:404-406, 1990), very large libraries can be constructed (10⁶-10⁸ chemical entities). A second approach uses primarily chemical methods, of which the Geysen method (Geysen et al., Molecular Immunology 23:709-715, 1986; Geysen et al. J. Immunologic Method 102:259-274, 1987; and the method of Fodor et al. (Science 251:767-773, 1991) are examples. Furka et al. (14th International Congress of Biochemistry, Volume #5, Abstract FR:013, 1988; Furka, Int. J. Peptide Protein Res. 37:487-493, 1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

In another aspect, synthetic libraries (Needels et al., Proc. Natl. Acad. Sci. USA 90:10700-4, 1993; Ohlmeyer et al., Proc. Natl. Acad. Sci. USA 90:10922-10926, 1993; Lam et al., International Patent Publication No. WO 92/00252; Kocis et al., International Patent Publication No. WO 9428028) and the like can be used to screen for MMP-2 ligands according to the present invention. Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., Tib Tech, 14:60, 1996).

Knowledge of the crystal structure of MMP-2 can provide an initial clue as the agonists or antagonists of the protein. Identification and screening of agonists is further facilitated by determining structural features of the protein, e.g., using X-ray crystallography, neutron diffraction, nuclear magnetic resonance spectrometry, and other techniques for structure determination.

In vivo screening methods. Intact cells or whole animals expressing a gene encoding MMP-2 can be used in screening methods to identify candidate drugs.

In one series of embodiments, a permanent cell line is established. Alternatively, cells (including without limitation mammalian, insect, yeast, or bacterial cells) are transiently programmed to express an MMP-2 gene by introduction of appropriate DNA or mRNA. Identification of candidate compounds can be achieved using any suitable assay, including without limitation (i) assays that measure selective binding of test compounds to MMP-2 (ii) assays that measure the ability of a test compound to modify (i.e., inhibit or enhance) a measurable activity or function of MMP-2 and (iii) assays that measure the ability of a compound to modify (i.e., inhibit or enhance) the transcriptional activity of sequences derived from the promoter (i.e., regulatory) regions the MMP-2 gene.

High-Throughput Screen. Agents according to the invention may also be identified by screening in high-throughput assays, including without limitation cell-based or cell-free assays. It will be appreciated by those skilled in the art that different types of assays can be used to detect different types of agents. Several methods of automated assays have been developed in recent years so as to permit screening of tens of thousands of compounds in a short period of time. Such high-throughput screening methods are particularly preferred.

### Activating MMP-2

Supplying a subject with an MMP-2 activator, such as one identified through the foregoing procedures, or a known activator such as MT1-MMP, can also lead to increased MMP-2 activity, partially or fully overcoming an MMP-2 deficiency.

In a specific embodiment, the invention contemplates administration of MT1-MMP protein, or a preferably a soluble variant thereof, gene therapy vector that expresses MT1-MMP, or other mechanisms for introducing MTI-MMP activity (see Yoshizaki et al., Oncol Rep. 9(3), 607-11 (2002); Toschi et al., Mol Cell Biol. 12(10), 2934-46; Nguyen et al., Int J. Biochem Cell Biol 32(6), 621-31 (2000)). In a further embodiment contemplates a combined approach of administering both MMP-2 (particularly by gene therapy) and increasing MT 1-MMP activity.

The MMP-2 proenzyme (progelatinase A), is constitutively expressed in a variety of cell types. These include osteoblasts and, to a lesser extent, osteoclasts in certain species (Murphy G et al., J Cell Sci 92, 487-95 (1989); Murphy G. et al., Biochim Biophys Acta 831, 49-58 (1985); Rifas L et al., J. Clin Invest 84, 686-94 (1989); Lorenzo J A et al., Matrix 12, 282-90 (1992); Meikle M C et al., J Cell Sci 103, 1093-9 (1992); Meikle M C et al., Bone 17, 255-60 (1995); Hill P A et al., J Cell Biochem 56, 118-30 (1994)) although the rate of synthesis in osteoclasts may be quite low (Dew G et al., Cell Tissues Res 299, 385-94 (2000)). Once translated, the cysteine-rich N-terminus of the progelatinase blocks proteolytic activity by interfering with the active site Zn⁺² ion—the critical “cysteine switch” mechanism (Van Wart H E. et al., Proc Natl Acad Sci USA 87, 5578-82 (1990)). Physiologic activation is regulated by a cell surface interaction and achieved following the formation of a tri-molecular complex between MMP-2, the membrane bound type I matrix metalloproteinase, MT1-MMP, and tissue inhibitor of metalloproteinase (TIMP)-2 (Butler G S et al., Eur J Biochem 244, 653-7 (1997)). In this model, the catalytic domain of MT1-MMP binds the N-terminal portion of TIMP-2. The MT1-MMP-bound TIMP-2 then acts as a molecular link providing its negatively charged C-terminus to bind the hemopexin-like domain of pro-MMP-2 (Strongin AY et al., J Biol Chem 270, 5331-8 (1995);Butler G S et al., J Biol Chem 273, 871-80 (1998)). Cleavage at Asn66-Leu77 in the MMP-2 prodomain by a free MT1-MMP molecule, generates an intermediate which is autocatalysed to produce fully active MMP-2.

In accord with this model, MT1-MMP (Holmbeck K et al., Cell 99, 81-92(1999) and TIMP-2 (Caterina J J et al., J. Biol Chem 275, 26416-22(2000)) deficient mice were each unable to completely activate pro-MMP-2. Of note, the specificity of the domains involved in this complex has recently been established using a variety of techniques such as site-directed mutagenesis and yeast two-hybrid analysis (Hernandez-Barrantes S et al., J. Biol Chem 275, 12080-9 (2000); Overall C M et al., J. Biol Chem 274, 4421-9 (1999); Overall C M et al., J Biol Chem 275, 39497-506(2000)).

Interestingly, MT1-MMP deficient mice display a marked skeletal phenotype which mimics MONA (Holmbeck K. et al., Cell 99, 81-92 (1999); Zhou Z. et al., Proc Natl Acad Sci USA 97, 4052-7 (2000). These mice had craniofacial dysmorphia, osteopenia, arthritis, dwarfism and soft-tissue fibrosis—a striking parallel to the human multicentric osteolysis syndromes, and in particular MONA. As would be expected, these mice and their cultured fibroblasts were unable to fully activate pro-MMP-2 (Holmbeck K et al., Cell 99, 81-92(1999). Since it was believed that MMP-2 “knockouts” lacked a phenotype, the skeletal consequences of MT1-MMP deletion have been ascribed solely to the loss of MT1-MMP. In the light of the MMP-2 human and mouse results disclosed here, this interpretation must now be re-examined.

These data clearly establish the ability of MT1-MMP activity to increase the level of MMP-2 activity, and overcome MMP-2 deficiencies.

### Suppressing MMP-2 Inhibitors

As noted above, it is also possible to suppress MMP-2 inhibitors, particularly the tissue inhibitor of metaloproteinase-2 (TIMP-2) (see Yoshizaki et al., supra; Mackay et al., Invasion Metastasis, 12(3-4), 168-84 (1992); ). Various techniques are available to inhibit TIMP-2, including phorbol esters and cytokines (Mackay et al., supra), anti-TIMP-2 antibodies, TIMP-2 antisense technology (for reduced expression; see, e.g., Okamoto et al, Mol Hum Reprod. 8(4):392-8 (2002).

TIMP-2 knockout mice also were deficient in pro-MMP-2 activation, and yet these mice appeared phenotypically normal and developed and procreated indistinguishably from wildtype littermates (Caterina J J, et al., J. Biol Chem 275, 26416-22 (2000); Caterina J. et al., Ann NY Acad Sci 878, 528-30 (1999)). Of note, no skeletal-investigations were undertaken.

TIMP-2, which helps to mediate cell-surface activation of pro-MMP-2 by binding to MT1-MMP, is also a direct inhibitor of active MMP-2. This inhibition is specifically mediated by TIMP-2's C-terminus, which binds to the N-terminal region of MMP-2 (Fridman R et al., J Biol Chem 267, 15398-405 (1992); Murphy A N et al., J. Cell Physiol 157, 351-8 (1993); Nguyen Q et al., A. Biochemistry 33, 2089-95 (1994)). It has been noted that higher TIMP-2 concentrations are required for inhibition than activation (Creemers L B et al., Matrix Biol 17, 35-46 (1998)). Thus, variations in the ratios of TIMP-2 influence the availability, and hence activity, of MMP-2.

### Pharmaceutical Compositions

The present invention also provides pharmaceutical compositions comprising an active ingredient (that can also be called a pharmaceutical agent herein) against a disease mediated by decreased MMP-2 function, with a pharmaceutically acceptable carrier.

In one embodiment of the invention the active ingredient is a vector or a nucleic acid that encodes an MMP-2 protein.

In another embodiment, it is an MMP-2 protein, a deletion mutant or a non-peptide analog thereof.

In a further embodiment, this active ingredient is a substance that stimulates MMP-2 activity, as described above, e.g., MT1-MMP or an MT1-MMP activator, or an inhibitor or TIMP-2.

The concentration or amount of the active ingredient depends on the desired dosage and administration regimen, as discussed below. Suitable dose ranges may include from about 1 mg/kg to about 100 mg/kg of body weight per day.

The pharmaceutical compositions may also include other biologically active compounds, including but by no means limited to, androgens, anabolic hormones, non-steroidal anti-inflammatory drugs, immunomodulatory drugs, etc. In a specific embodiment, the compositions do not include androgens or anabolic hormones (and, indeed, in a related specific embodiment, such compounds are not administered with the estrogen compounds).

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

A composition comprising “A” (where “A” is a single protein, DNA molecule, vector, recombinant host cell, etc.) is substantially free of “B” (where “B” comprises one or more contaminating proteins, DNA molecules, vectors, etc.) when at least about 75% by weight of the proteins, DNA, vectors (depending on the category of species to which A and B belong) in the composition is “A” . Preferably, “A” comprises at least about 90% by weight of the A+B species in the composition, most preferably at least about 99% by weight. It is also preferred that a composition, which is substantially free of contamination, contain only a single molecular weight species having the activity or characteristic of the species of interest.

According to the invention, the pharmaceutical composition of the invention can be introduced parenterally, transmucosally, e.g., orally (per os), nasally, or rectally, or transdermally. Parental routes include intravenous, intra-arteriole, intramuscular, intradermal, subcutaneous, intraperitoneal, intraventricular, and intracranial administration. Preferably, administration is topical.

In another embodiment, the active ingredient can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.). To reduce its systemic side effects, this may be a preferred method for introducing the agent.

In yet another embodiment, the therapeutic compound can be delivered in a controlled release system. For example, a polypeptide may be administered using intravenous infusion with a continuous pump, in a polymer matrix such as poly-lactic/glutamic acid (PLGA), a pellet containing a mixture of cholesterol and the active ingredient (SilasticR™; Dow Corning, Midland, Mich.; see U.S. Pat. No. 5,554,601) implanted subcutaneously, an implantable osmotic pump, a transdermal patch, liposomes, or other modes of administration. In one embodiment, a pump maybe used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)).

### Diagnostics

The present invention further encompasses a method for diagnosing a disease mediated by decreased MMP-2 function.

As used herein, the term “diagnosis” refers to the identification of the disease at any stage of its development, and also includes the determination of a predisposition of a subject to develop the disease.

The method of the invention comprises assessing the level of expression or activity of MMP-2 in a biological sample of a test subject and comparing it to the level of expression or activity of MMP-2 in a control sample. These nucleic acid based assays and protein based assays are discussed in greater detail below.

A “biological sample” is any body tissue or fluid likely to contain MMP-2 protein or mRNA. Such samples preferably include blood or a blood component (serum, plasma).

The “level of expression of MMP-2” refers either to the quantity of mRNA that is expressed by the test subject and that encodes a MMP-2 protein or to the quantity of MMP-2 protein produced by the test-subject. The “activity of MMP-2” refers to the biological properties of the enzyme as described above.

Noteworthy different MMP-2 mutations imply different levels of expression or activity of the protein, and different associated symptoms or different degrees of severity of the disease, as described in greater detail in the Examples below.

The components useful in practicing the diagnostic and prognostic aspects of the invention can be conveniently provided in kit form, as set forth in greater detail below. Such kits contain, at least, a detection assay for inactivation of MMP-2.

### Nucleic Acid Assays and Kits

Nucleic acid assays for MMP-2 inactivation are based on detection of mutations or modifications in the MMP-2 gene that result in its inactivation. The DNA may be obtained from any cell source. Non-limiting examples of cell sources available in clinical practice include without limitation blood cells, buccal cells, cervicovaginal cells, epithelial cells from urine, fetal cells, or any cells present in tissue obtained by biopsy. Cells may also be obtained from body fluids, including without limitation blood, plasma, serum, lymph, milk, cerebrospinal fluid, saliva, sweat, urine, feces, and tissue exudates (e.g., pus) at a site of infection or inflammation. DNA is extracted from the cell source or body fluid using any of the numerous methods that are standard in the art. It will be understood that the particular method used to extract DNA will depend on the nature of the source. Generally, the minimum amount of DNA to be extracted for use in the present invention is about 25 pg (corresponding to about 5 cell equivalents of a genome size of 4×10⁹ base pairs).

Mutations of the MMP-2 genomic DNA include an insertion in the gene, deletion of the gene, truncation of the gene (e.g., due to a nonsense, missense, or frameshift mutation), or disregulation of gene expression (e.g., due to a frameshift mutation or a splice-site mutation). The identification of several specific mutations is described in the Examples below. Identification of gene deletion is readily accomplished using nucleic acid probes, PCR analysis, or direct DNA sequencing. Determination of polymorphic positions is achieved by any means known in the art, including but not limited to direct sequencing, hybridization with allele-specific oligonucleotides, allele-specific PCR, ligase-PCR, HOT cleavage, denaturing gradient gel electrophoresis (DGGE), and single-stranded conformational polymorphism (SSCP). Denaturing high performance liquid chromatography (DHPLC) may also be a convenient qualitative technique to screen for the presence of mutations or polymorphims. DHPLC is a highly sensitive PCR-based technique for nucleotide variant detection which relies on the principle of heteroduplex analysis by ion-pair reverse-phase liquid chromatography under partially denaturing conditions (Liu et al., Nuc Acids Res. 1998, 26:1396-400; O'Donovan et al. Genomics. 1998, 52:44-9). Direct sequencing may be accomplished by any method, including without limitation chemical sequencing, using the Maxam-Gilbert method; by enzymatic sequencing, using the Sanger method; mass spectrometry sequencing; and sequencing using a chip-based technology (see, e.g., Little et al., Genet. Anal., 1996, 6:151). Preferably, DNA from a subject is first amplified bypolymerase chain reaction (PCR) using specific amplification primers.

Gene expression, or lack of gene expression, can be directly evaluated by detecting MMP-2 mRNA. Methods for detecting mRNA include Northern blotting and reverse transcriptase (RT)-PCR. These methods can be used to determine whether or not expression occurs, and whether a truncated (or oversized) message is expressed. All of these factors can help establish inactivation of MMP-2.

A nucleic acid assay kit of the invention will comprise a nucleic acid that specifically hybridizes under stringent conditions to a MMP-2 gene, and an assay detector, e.g., a label. Where the kit is a PCR-based kit, a primer pair will be included; in this case, the detector may simply be a reagent such as ethidium bromide to quantify amplified DNA. Optional components include buffer or buffer reagents, nucleotides, and instructions for use of the kit. If possible, a positive control is also included, e.g., a probe or primer pair for an endogenously expressed gene, such as actin or tubulin.

### Mutational Analysis Using Microarray Technology

The present invention further makes use of microarrays for identifying mutations in the MMP-2 gene, more particularly SNPs (single nucleotide polymorphisms).

Such microarrays are well known in the art (see for example the following: U.S. Pat. Nos. 6,045,996; 6,040,138; 6,027,880; 6,020,135; 5,968,740; 5,959,098; 5,945,334; 5,885,837; 5,874,219; 5,861,242; 5,843,655; 5,837,832; 5,677,195 and 5,593,839). The microarray techniques developed by Affymetrix may be particularly useful in that request.

The solid support on which oligonucleotides are attached may be made from glass, silicon, plastic (e.g., polypropylene, nylon), polyacrylamide, nitrocellulose, or other materials.

One method for attaching the nucleic acids to a surface is by printing on glass plates, as is described generally by Schena et al., Science 1995, 270:467-470. This method is especially useful for preparing microarrays of cDNA. See also DeRisi et al., Nature Genetics 1996, 14:457-460, ; Shalon et al., Genome Res. 1996, 6:639-645; and Schena et al., Proc. Natl. Acad. Sci. USA 1995, 93:10539-11286.

Another method of making microarrays is by use of an inkjet printing process to bind genes or oligonucleotides directly on a solid phase, as described, e.g., in U.S. Pat. No. 5,965,352.

Other methods for making microarrays, e.g., by masking (Maskos and Southern, Nuc. Acids Res. 1992, 20:1679-1684), also may be used. In principal, any type of array, for example, dot blots on a nylon hybridization membrane (see Sambrook et al., Molecular Cloning A Laboratory Manual (2nd Ed.), Vol. 1-3, Cold Spring Harbor Laboratory, Cold Spring Harbor, N.Y., 1989) could be used, although, as will be recognized by those of skill in the art, very small arrays will be preferred because hybridization volumes will be smaller. Nucleic acid hybridization and wash conditions are chosen so that the attached oligonucleotides “specifically bind” or “specifically hybridize” to at least a portion of the MMP-2 gene present in the tested sample, i.e., the probe hybridizes, duplexes or binds to the MMP-2 locus with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra, and Chee et al., Science 1996, 274:610-614).

Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., Current Protocols in Molecular Biology, Greene Publishing and Wiley-Interscience, New York, 1987. When the cDNA microarrays of Schena et al. are used, typical hybridization conditions are 5×SSC plus 0.2% SDS at 65° C. for 4 hours followed by washes at 25° C. in low stringency wash buffer (1×SSC plus 0.2% SDS) followed by 10 minutes at 25° C. in high stringency wash buffer (0.1×SSC plus 0.2% SDS) (Shena et al., Proc. Natl. Acad. Sci. USA 1996, 93:10614). Useful hybridization conditions are also provided in, e.g., Tijessen, 1993, Hybridization With Nucleic Acid Probes, Elsevier Science Publishers B. V. and Kricka, 1992, Nonisotopic DNA Probe Techniques, Academic Press San Diego, Calif.

A variety of methods are available for detection and analysis of the hybridization events. Depending on the reporter group (fluorophore, enzyme, radioisotope, etc.) used to label the DNA probe, detection and analysis are carried out fluorimetrically, calorimetrically or by autoradiography. By observing and measuring emitted radiation, such as fluorescent radiation or a particle emission, information may be obtained about the hybridization events.

When fluorescently labeled probes are used, the fluorescence emissions at each site of transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al. Genome Res. 1996, 6:639-695).

Signals are recorded and, in a preferred embodiment, analyzed by computer, e.g., using a 12 bit analog to digital board. In one embodiment the scanned image is despeckled using a graphic program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

### Protein Based Assays

As an alternative to analyzing MMP-2 nucleic acids, one can evaluate MMP-2 on the basis of protein expression. Indeed, this assay may be more informative, since MMP-2 mRNA levels may appear high, but a mutation in the sequence may make the mRNA less effective for translation, resulting in reduction or elimination of protein expression.

In a preferred embodiment, MMP-2 is detected by immunoassay. For example, Western blotting permits detection of the presence or absence of MMP-2. Other immunoassay formats can also be used in place of Western blotting, as described below for the production of antibodies. One of these is ELISA assay.

In ELISA assays, an antibody against an MMP-2 polypeptide is immobilized onto a selected surface, for example, a surface capable of binding proteins such as the wells of a polystyrene microtiter plate. After washing to remove incompletely adsorbed antibodies, a nonspecific protein such as a solution of bovine serum albumin (BSA) may be bound to the selected surface. This allows for blocking of nonspecific adsorption sites on the immobilizing surface and thus reduces the background caused by nonspecific binding of polypeptides onto the surface. The immobilizing surface is then contacted with a sample, such as clinical or biological materials, to be tested in a manner conductive to immune complex (antigen/antibody) formation. This may include diluting the sample with diluents, such as solutions of BSA, bovine gamma globulin (BGG) and/or phosphate buffered saline (PBS)/Tween. The sample is then allowed to incubate for from 2 to 4 hours, at temperatures such as of the order of about 25° to 37° C. Following incubation, the sample-contacted surface is washed to remove non-immunocomplexed material. The washing procedure may include washing with a solution, such as PBS/Tween or borate buffer. Following formation of specific immunocomplexes between the test sample and the bound antibody, and subsequent washing, the occurrence, and an even amount of immunocomplex formation may be determined by subjecting the immunocomplex to a second antibody having specificity for a different epitope of MMP-2 protein. To provide detecting means, the second antibody may have an associated activity such as an enzymatic activity that will generate, for example, a color development upon incubating with an appropriate chromogenic substrate. Quantification may then be achieved by measuring the degree of color generation using, for example, a visible spectra spectrophotometer.

Alternatively, a biochemical assay can be used to detect expression of MMP-2, e.g., by the presence or absence of a band by polyacrylamide gel electrophoresis; by the presence or absence of a chromatographic peak by any of the various methods of high performance liquid chromatography, including reverse phase, ion exchange, and gel permeation; by the presence or absence of MMP-2 in analytical capillary electrophoresis chromatography, or any other quantitative or qualitative biochemical technique known in the art.

For both kinds of assays, biopsy tissue is obtained from a subject. Antibodies that are capable of binding to MMP-2 are then contacted with samples of the tissue under conditions that permit antibody binding to determine the presence or absence of MMP-2. In a further embodiment, antibodies that distinguish polymorphic variants of MMP-2 can be used. The antibodies may be polyclonal or monoclonal, preferably monoclonal. Measurement of specific antibody binding to cells may be accomplished by any known method, e.g., quantitative flow cytometry, or enzyme-linked or fluorescence-linked immunoassay. The presence or absence of a particular mutation, and its allelic distribution (i.e., homozygosity vs. heterozygosity) is determined by comparing the values obtained from a patient with norms established from populations of patients having known polymorphic patterns.

The components for detecting MMP-2 protein can be conveniently provided in a kit form. In its simplest embodiment, a kit of the invention provides a MMP-2 detector, e.g., a detectable antibody (which may be directly labeled or which may be detected with a secondary labeled reagent).

These immunoassays discussed above involve using antibodies directed against the MMP-2 protein or fragments thereof. The production of such antibodies is described below.

Anti-MMP-2 Antibodies

Such antibodies include but are not limited to polyclonal, monoclonal, chimeric, single chain, Fab fragments, and an Fab expression library.

Various procedures known in the art may be used for the production of polyclonal antibodies to MMP-2 polypeptides or derivative or analog thereof. For the production of antibody, various host animals can be immunized by injection with the antigenic polypeptide, including but not limited to rabbits, mice, rats, sheep, goats, etc. Preferably, the immunized animal is of the same species as the animal who will receive the antibodies in passive immunization, to avoid allergic reactions to the antibodies.

For preparation of monoclonal antibodies directed toward the MMP-2 polypeptides, any technique that provides for the production of antibody molecules by continuous cell lines in culture may be used. These include but are not limited to the hybridoma technique originally developed by Kohler and Milstein (Nature 256:495-497, 1975), as well as the trioma technique, the human B-cell hybridoma technique (Kozbor et al., Immunology Today 4:72, 1983; Cote et al., Proc. Natl. Acad. Sci. U.S.A. 80:2026-2030, 1983), and the EBV-hybridoma technique to produce human monoclonal antibodies (Cole et al., in Monoclonal Antibodies and Cancer Therapy, Alan R. Liss, Inc., pp. 77-96, 1985). In an additional embodiment of the invention, monoclonal antibodies can be produced in germ-free animals (International Patent Publication No. WO 89/12690, published Dec. 28, 1989).

According to the invention, techniques described for the production of single chain antibodies (U.S. Pat. Nos. 5,476,786 and 5,132,405 to Huston; U.S. Pat. No. 4,946,778) can be adapted to produce the MMP-2 polypeptide-specific single chain antibodies. Indeed, these genes can be delivered for expression in vivo. An additional embodiment of the invention utilizes the techniques described for the construction of Fab expression libraries (Huse et al., Science 246:1275-1281, 1989) to allow rapid and easy identification of monoclonal Fab fragments with the desired specificity for an MMP-2 polypeptide, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

MMP-2 Activity Assays

The level of activity of MMP-2 may be determined in vtro or in vivo by any standard technique well-known by one skilled in the art.

Exemplary techniques for identifying the MMPs responsible for the collagenolytic activity within tissues fall into two categories: SDS-PAGE-zymography and labeled substrate release. The most widely used zymographic technique is gelatin zymography. Zymography is a single step staining method for quantitation of proteolytic activity on substrate gels. In using this technique, samples are electrophoresed on a gelatin zymogram gel. Such gels may be available from a variety of commercial sources such as, but not limited to Novex. The gels are developed according to the manufacturer's instructions and quantitated.

Collagen lattice assays also may be used to assess MMP activity. Cultured fibroblasts incorporated into type I collagen lattices share certain characteristics with fibroblasts within dermis and may be used as an in vitro model of wound contraction. Fibroblast morphology and organization within the lattice and lattice contraction are studied over time. Briefly, cells are cultured within and on top of collagen lattices. Lattice diameter is measured at predetermined intervals, preferably every hour for the first seven hours and everyday afterwards, using any method known in the art. In one example, a stereomicroscope is used to measure the degree of contraction. Cellular morphology and organization is studied by fixing and processing gels. Sections also may be stained, such as with hematoxylin and eosin, prior to microscopic examination.

In collagen dissolution assays are skin fibroblasts are seeded in contact with a reconstituted film of type I collagenfibrils. Briefly, plates are coated with a type I collagen fibrils, and a pellet of cells in growth medium is seeded into the center of each well. Fibroblasts are allowed to attach and then washed. Degradation of type I collagen can then be measured. Degradation can be evaluated in a basal state and following induction of MMP expression by any method known in the art such as, but not limited to, TNFα/IL-1 and phorbolester.

Binding of MMP-2 protein to integrin α5β3, or to MCP-3 protein, may be assayed by various binding assays.

### Modulation of Hair growth

The present invention further demonstrates that the patients suffering from the MONA syndrome and showing a deficiency of MMP-2 are hirsute. This provides a basis for treating hirsutism by stimulating MMP-2 activity, as described above, but also for any desired removal of hair in a subject. Alternatively this provides a basis for the prevention or treatment of baldness or alopecia in a subject, by inhibiting MMP-2 activity.

### Removal of Hair

The present invention contemplates a method for removal of hair in a subject, which method comprises stimulating MMP-2 activity in the subject. Preferably this method comprises administering a substance that stimulates MMP-2 activity. This method encompasses cosmetic applications, and the substance may be preferably in the form of a cosmetic composition, with a cosmetically acceptable carrier. Any of the substances that may be identified by the screening method as described above may be useful for that purpose.

According to the present invention unwanted mammalian (including human) hair growth can be reduced by administering to the subject, preferably by applying to the skin, a composition including a substance that stimulates MMP-2 in an amount effective to reduce hair growth.

The unwanted hair growth which is reduced may be normal hair growth, or hair growth that results from an abnormal or diseased condition.

The composition may be topically applied to a selected area of the body from which it is desired to reduce hair growth. For example, the composition can be applied to the face, particularly to the beard area of the face, e.g., the cheek, neck, upper lip, chin, etc. The composition can also be applied to the legs, arms, torso or armpits. The composition is particularly suitable for reducing the growth of unwanted hair in women suffering from hirsutism or other conditions. In humans, the composition should be applied once or twice a day, or even more frequently, for at least three months to achieve a perceived reduction in hair growth. Reduction in hair growth is demonstrated when the frequency or hair removal is reduced, or the subject perceives less hair on the treated site, or quantitatively, when the weight of hair removed by shaving (i.e., hair mass) is reduced.

### Treatment of Baldness or Alopecia

The invention further provides a method for the prevention or treatment of baldness or alopecia in a subject, which method comprises administering to the subject in need of such treatment an effective amount of a substance that inhibits MMP-2 activity.

Both direct and indirect inhibitors of MMPs are known. One form of indirect inhibition of MMPs involves stimulating an increase in the expression or catalytic activity of endogenous tissue-derived inhibitors of MMP. Known indirect inhibitors that apparently act via this mechanism include bromo-cyclic adenosine monophosphate; protocatechuic aldehyde (3,4-dihydroxybenzaldehyde); and estramustine (estradiol-3-bis(2-chloroethyl)carbamate). Examples of inhibitors of an MMP include 1,10-phenanthroline (o-phenanthroline); batimastat also known as BB-94; 4-(N-hydroxyamino)-2R-isobutyl-3S-(thiopen-2-ylthiomethyl)-succinyl-L-ph enylalanine-N-methylamidecarboxyalkylamino-based compounds such as N-1-(R)-carboxy-3-(1,3-dihydro-2H-benzsoindol-2-yl)propyl-N′,N′-dimethyl-L-leucinamide, trifluoroacetate (J. Med Chem. 36:4030-4039, 1993); marimastat (BB-2516); N-chlorotaurine; eicosapentaenoic acid; matlystatin-B; actinonin (3-1-2-(hydroxymethyl)-1-pyrolidinylcarbamoyl-octanohydroxamic acid); N-phosphonalkyl dipeptides such as N-N-((R)-1-phosphonopropyl)-(S)-leucyl-(S)-phenylalanine-N-methylamide (J. Med. Chem. 37:158-169, 1994); peptidyl hydroxamic acids such as pNH₂-Bz-Gly-Pro-D-Leu-D-Ala-NHOH (Biophys. Biochem. Res. Comm. 199: 1442-1446, 1994); Ro-31-7467, also known as 2-(5-bromo-2,3-dihydro-6-hydroxy-1,3-dioxo-1H-benzdeisoquinolin-2-yl)methyl(hydroxy)-phosphinyl-N-(2-oxo-3-azacyclotridecanyl)-4-methylval eramide; CT1166, also known as N1[N-2-(morpholinosulphonylamino)-ethyl-3-cyclohexyl-2-(S)-propanamidyl]-N 4-hydroxy-2-(R)-3-(4-methylphenyl)propyl-succinamide (Biochem. J. 308:167-175, 1995); bromocyclic-adenosine monophosphate; protocatechuic aldehyde (3,4-dihydroxybenzaldehyde); estramustine (estradiol-3-bis(2-chloroethyl)carbamate); tetracycline (4-(dimethylamino)-1,4,4a,5,5a,6,11,12a-octahydro-3,6,10,12,12a-pentahydroxy-6-methyl-1,11-dioxo-2-naphthacenecarboxamide); minocycline (7-dimethylamino-6-dimethyl-6-deoxytetracycline); methacycline (6-methylene oxytetracycline); and doxycycline (α-6-deoxy-5-hydroxytetracycline). Preferably, the inhibitor of MMP-2 includes an inhibitor other than an unsaturated fatty acid such as eicosapentaenoic acid.

Alternatively the inhibitor of MMP-2 that is used is a new inhibitor identified by any screening test similar to the screening methods as above described.

The inhibitors of the MMP preferably are incorporated in a topical composition that preferably includes a non-toxic dermatologically acceptable vehicle or carrier which is adapted to be spread upon the skin. Examples of suitable vehicles are acetone, alcohols, or a cream, lotion, or gel which can effectively deliver the active compound. In addition, a penetration enhancer may be added to the vehicle to further enhance the effectiveness of the formulation.

The concentration of the inhibitor in the composition may be varied over a wide range up to a saturated solution, preferably from 0.1% to 30% by weight or even more; the reduction of hair growth increases as the amount of inhibitor applied increases per unit area of skin. The maximum amount effectively applied is limited only by the rate at which the inhibitor penetrates the skin. The effective amounts may range, for example, from 10 to 3000 micrograms or more per square centimeter of skin.

### Cosmetic Compositions

The stimulating or inhibiting substances as described above may be advantageously formulated in a cosmetic composition, that comprises such substance with a cosmetically acceptable carrier.

The cosmetic compositions according to the invention preferably contain a cosmetically acceptable aqueous medium. They have a pH which can range from 3.5 to 11, preferably between 5.5 and 11 and even more preferably between 5.5 and 8.5. The cosmetically acceptable medium for the compositions according to the invention consists more particularly of water and optionally of cosmetically acceptable organic solvents.

The organic solvents can represent from 0.5 to 90% of the total weight of the composition. They can be chosen from the group consisting of hydrophilic organic solvents, lipophilic organic solvents, amphiphilic solvents or mixtures thereof. Among the hydrophilic organic solvents, mention may be made, for example, of linear or branched lower monoalcohols having from 1 to 8 carbon atoms, polyethylene glycols having from 6 to 80 ethylene oxide units, and polyols.

As amphiphilic organic solvents, mention may be made of polypropylene glycol (PPG) derivatives, such as esters of polypropylene glycol and of fatty acid, derivatives of PPG and of fatty alcohol, such as PPG-23 oleyl ether, and PPG-36 oleate. As lipophilic organic solvents, mention may be made, for example, of fatty esters such as diisopropyl adipate, dioctyl adipate, alkyl benzoates and dioctyl malate.

In order for the cosmetic compositions of the invention to be more pleasant to use (softer when applied, more nourishing and more emollient), it is possible to add a fatty phase to the medium of these compositions.

The fatty phase can represent up to 50% of the total weight of the composition. This fatty phase can contain an oil or a wax or mixtures thereof, and can also comprise fatty acids, fatty alcohols and fatty acid esters. The oils can be chosen from animal, plant, mineral or synthetic oils and in particular from liquid petroleum jelly, liquid paraffin, isoparaffins, poly-α-olefins, fluoro oils and perfluoro oils. Similarly, the waxes can be chosen from animal, fossil, plant, mineral or synthetic waxes which are known per se.

The compositions of the invention can contain adjuvants that are common in the cosmetics field, such as other standard gelling agents and/or thickeners; emulsifiers; surfactants; moisturizers; emollients; hydrophilic or lipophilic active agents such as ceramides; anti-free-radical agents; sequestering agents; antioxidants; preserving agents; acidifying or basifying agents; fragrances; fillers; dyestuffs; modified or non-modified, volatile or non-volatile silicones; reducing agents. The amounts of these various adjuvants are those used conventionally in the fields considered.

The compositions according to the invention can be in any form which is suitable for topical application, in particular in the form of a thickened lotion, in the form of aqueous or aqueous-alcoholic gels, in the form of vesicle dispersions or in the form of simple or complex emulsions (O/W, W/O, O/W/O or W/O/W emulsions) and can be of liquid, semi-liquid or solid consistency, such as creams, milks, gels, cream-gels, pastes and sticks, and can optionally be packaged as an aerosol and can be in the form of mousses or sprays. These compositions are prepared according to the usual methods.

### EXAMPLES

The following examples illustrate the invention, but are not limiting.

### Example 1

### Identification of MMP-2 Mutations that Cause a Multicentric Osteolysis and Arthritis Syndrome

### Materials and Methods

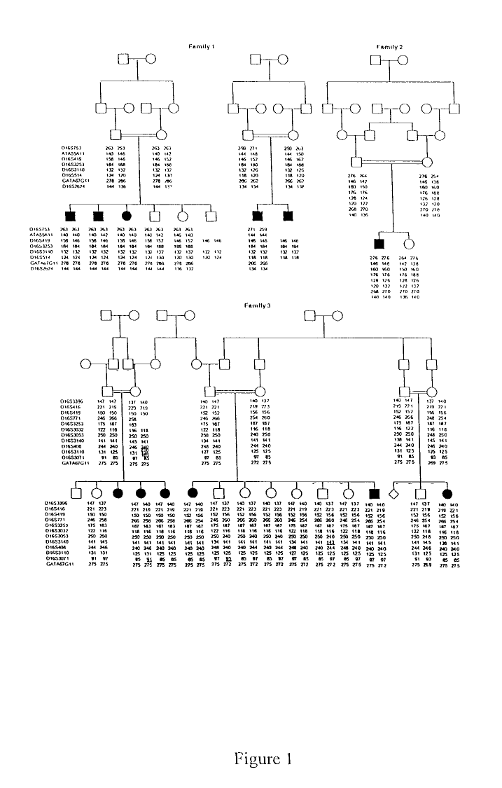

Saudi family members. An autosomal recessive form of multicentric osteolysis with carpal and tarsal resorption, crippling arthritic changes, marked osteoporosis, palmar and plantar subcutaneous nodules, and distinctive facies was recently described in a number of consanguineous Saudi Arabian families (Al-Mayouf, et al., Am. J. Med. Genet. 93, 5-10 (2000); Al Aqeel, A. et al., Am. J. Med. Genet. 93, 11-18 (2000)). Samples used herein comprised genomic DNAs obtained from 11 affected and 24 unaffected members of four Saudi families (See FIG. 1). Genomic DNA from blood samples was obtained from all Saudi family members with informed consent.

Linkage analysis. Three pooled samples were then generated: parents, unaffected siblings, and affected siblingss such that the final genomic DNA concentration was equal to 10 ng/μl. Fluorescently-labeled microsatellite markers from the Human Screening Panel, Version 9.0 (Research Genetics) were used for amplification and additional markers were obtained to further define the critical region. PCR products were then electrophoretically separated on an ABI 377 DNA Sequencer (Perkin-Elmer) and the data were analyzed using the Genescan and Genotyper Programs (Perkin-Elmer). Multipoint parametric linkage analysis was performed using GeneHunter linkage-analysis software (Kruglyak, L. et al, Am. J. Hum. Genet. 58, 1347-1363 (1996)). Linkages were calculated assuming a 100%-penetrant autosomal recessive trait, with a disease gene frequency of 0.0001. Equal allele frequencies for each microsatellite marker and equal recombination frequencies for males and females also were assumed. Sex-averaged genetic recombination maps were used to derive the intermarker distances.

DNA sequencing and analysis. Thirteen exons and the flanking 5′ promoter and 3′ untranslated regions of MMP-2 (Huhtala, P. et al., Genomics 6, 554-559 (1990)) from each family member were PCR-amplified using DNA primers. The PCR products were purified (Qiagen) and sequenced in both directions using the dRhodamine Dye Terminator Sequencing Kit (Perkin-Elmer). The resulting sequences were analyzed using the Sequencher 3.1 program (GeneCodes).

Cell culture and zymography. Fibroblast cultures were established using standard procedures and these were then grown in DMEM containing 10% fetal calf serum until cells were approximately 80% confluent. The media was removed, cells rinsed several times with serum-free media, and then grown an additional 36 hours prior to media collection and assay. Serum samples were collected and filtered through Centricon YM-10 filters (Amicon) for desalting and removal of low molecular weight proteins. Protein concentrations were determined by the Bradford assay. Serum-free conditioned media and serum samples were incubated for 10 min with non-reducing tris-glycine sample buffer with 2% SDS. Samples were electrophoresed (5 μl; 0.2 mg/ml protein) in 10% gelatin zymogram gels (Novex) and developed overnight according to the manufacturer's instructions. Gels were stained with Coomassie blue. All experiments were performed in duplicate at least twice.

### Results and Discussion

To identify the gene underlying this osteolysis/arthritis syndrome, a genome-wide search was performed for regions homozygous-by-descent using PCR-based microsatellite markers. Based on an initial genetic screen using families 1 and 2, a linkage for marker D16S3253 on chromosome 16q12 with a maximal LOD score of 4.59 at θ=0. Additional markers in this region, as defined by the 1999 Marshfield Map (Broman, K. W., et al., Am. J. Hum. Genet. 63, 861-869 (1998), were then analyzed and the multipoint LOD scores for the identified region of homozygosity were determined.

Results are presented in Table 1, below.

TABLE 1
Linkage Analysis

| | | | | | Maximum LOD |
|---|---|---|---|---|---|
| | | Maximum LOD | Maximum LOD | Maximum LOD | Fam |
| Marker | Distance (KcM) | (Fam 100) | (Fam 101) | (Fam 45) | (100 + 101 + 45) |
| D16S753 | 0.00 | 0.36 | 0.81 | −10000.00 | −9998.83 |
| | 0.83 | 0.28 | 0.80 | 1.02 | 2.10 |
| | 1.67 | 0.18 | 0.80 | 1.33 | 2.31 |
| | 2.50 | 0.03 | 0.81 | 1.52 | 2.36 |
| | 3.34 | −0.24 | 0.81 | 1.65 | 2.22 |
| ATA55A11 | 4.17 | −10000.00 | 0.82 | 1.76 | −9997.42 |
| | 5.22 | 1.06 | 0.82 | 1.76 | 3.64 |
| | 6.28 | 1.24 | 0.81 | 1.76 | 3.81 |
| | 7.33 | 1.24 | 0.80 | 1.76 | 3.80 |
| | 8.38 | 1.07 | 0.79 | 1.77 | 3.63 |
| D16S419 | 9.44 | −1.62 | 0.79 | 1.77 | 0.94 |
| | 10.49 | 1.44 | 0.76 | 1.77 | 3.97 |
| | 11.54 | 1.74 | 0.73 | 1.77 | 4.24 |
| | 12.60 | 1.92 | 0.70 | 1.77 | 4.39 |
| | 13.65 | 2.05 | 0.68 | 1.78 | 4.51 |
| D16S3253 | 14.71 | 2.15 | 0.66 | 1.78 | 4.59 |
| | 15.32 | 2.18 | 0.54 | 1.78 | 4.50 |
| | 15.94 | 2.20 | 0.39 | 1.78 | 4.37 |
| | 16.56 | 2.22 | 0.20 | 1.78 | 4.20 |
| | 17.18 | 2.24 | −0.12 | 1.78 | 3.90 |
| D16S3110 | 17.80 | 2.26 | −10000.00 | 1.78 | −9995.96 |
| | 19.08 | 2.30 | −2.18 | 1.77 | 1.89 |
| | 20.36 | 2.34 | −1.84 | 1.76 | 2.26 |
| | 21.63 | 2.37 | −1.73 | 1.76 | 2.40 |
| | 22.91 | 2.41 | −1.80 | 1.76 | 2.37 |
| D16S514 | 24.19 | 2.45 | −2.95 | 1.76 | 1.26 |
| | 24.81 | 2.45 | −2.24 | 1.76 | 1.97 |
| | 25.43 | 2.45 | −2.10 | 1.76 | 2.11 |
| | 26.05 | 2.46 | −2.10 | 1.76 | 2.12 |
| | 26.67 | 2.46 | −2.24 | 1.75 | 1.97 |
| GATA67G11 | 27.28 | 2.47 | −2.89 | 1.75 | 1.33 |
| | 27.90 | 2.37 | −1.15 | 1.74 | 2.96 |
| | 28.52 | 2.24 | −0.85 | 1.73 | 3.12 |
| | 29.14 | 2.07 | −0.68 | 1.72 | 3.11 |
| | 29.76 | 1.77 | −0.55 | 1.71 | 2.93 |
| D16S2624 | 30.38 | −10000.00 | −0.46 | 1.70 | −9998.76 |

By one method, heterozygosity and haplotype analysis defined the 13 cM interval between markers D16S3396 and GATA67G11 as the critical region. Independently from the above procedure, family 3 was mapped to establish a maximum multipoint LOD score of 1.61 at =0 for marker D16S3140, which was present within this region (Table 2). Haplotype analysis of Family 3 allowed for refined localization of the disease locus to a 1.2 cM region, flanked telomerically by marker D16S3140 and centromerically by marker D16S3032 (FIG. 1). The genetic position of the osteolysis/arthritis gene was localized to 16q12-21 on the cytogenetic map by reference to the CHLC Integrated Map ver8c7 (Sheffield, V. C. et al., Hum. Mol. Genet. 4, 1837-1844 (1995)).

TABLE 2
Linkage Analysis

| Marker | Distance (KcM) | Maximum LOD |
|---|---|---|
| D16S3396 | 0.00 | −2.65 |
| | 0.41 | −1.71 |
| | 0.82 | −1.54 |
| | 1.22 | −1.53 |
| | 1.63 | −1.69 |
| D16S416 | 2.04 | −2.65 |
| | 2.45 | −0.92 |
| | 2.86 | −0.64 |
| | 3.27 | −0.48 |
| | 3.67 | −0.37 |
| D16S419 | 4.08 | −0.29 |
| | 4.70 | −0.35 |
| | 5.32 | −0.44 |
| | 5.94 | −0.57 |
| | 6.56 | −0.84 |
| D16S771 | 7.18 | −2.68 |
| | 7.48 | −1.89 |
| | 7.79 | −1.74 |
| | 8.09 | −1.74 |
| | 8.39 | −1.89 |
| D16S3253 | 8.70 | −2.70 |
| | 9.00 | −1.88 |
| | 9.31 | −1.73 |
| | 9.61 | −1.73 |
| | 9.92 | −1.88 |
| D16S3032 | 10.22 | −2.71 |
| | 10.42 | 0.87 |
| | 10.63 | 1.17 |
| | 10.83 | 1.35 |
| | 11.03 | 1.47 |
| D16S3053 | 11.23 | 1.57 |
| | 11.28 | 1.58 |
| | 11.33 | 1.58 |
| | 11.38 | 1.59 |
| | 11.43 | 1.60 |
| D16S3140 | 11.48 | 1.61 |
| | 11.53 | 1.51 |
| | 11.58 | 1.38 |
| | 11.63 | 1.21 |
| | 11.68 | 0.91 |
| D16S408 | 11.73 | −10000.00 |
| | 11.78 | −4.37 |
| | 11.83 | −4.19 |
| | 11.88 | −4.19 |
| | 11.93 | −4.36 |
| D16S3110 | 11.98 | −10000.00 |
| | 12.21 | −3.07 |
| | 12.43 | −2.89 |
| | 12.65 | −2.89 |
| | 12.87 | −3.06 |
| D16S3071 | 13.10 | −10000.00 |
| | 13.40 | −0.89 |
| | 13.71 | −0.59 |
| | 14.01 | −0.43 |
| | 14.31 | −0.31 |
| GATA67G11 | 14.62 | −0.22 |

Inspection of the genes mapped to this region revealed several disease candidates including the MMP-2 gene—a member of the mammalian extracellular neutral metalloproteinases that degrade matrix proteins and are important mediators of connective tissue remodelling.

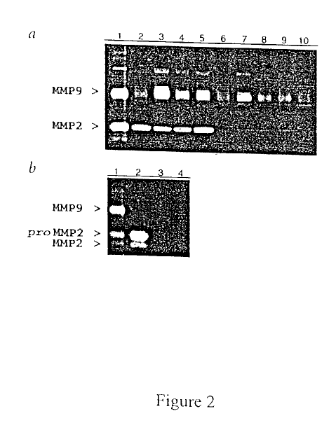

As shown in FIG. 2A, MMP-2 activity was not detected by zymography in serum samples from affected individuals of all four families. By contrast, serum MMP-9 (gelatinase B) activity, used as an internal control, was similar in the affected individuals and normal controls. Similarly, cultured skin fibroblasts (which have high MMP-2 activity) from an affected individual in Family 3 had no detectable MMP-2 activity by zymography (FIG. 2B).

Efforts were then directed to determine if these MMP-2 deficient families had the same or different mutations; the latter being predicted based upon their different haplotypes (see FIG. 1). In Family 3, all affected individuals were homoallelic for a nonsense mutation (TCA→TAA) in codon 244 of exon 5, predicting the replacement of a tyrosine residue by a stop codon in the first fibronectin type II domain (Y244X). Comparison of the mutant protein with the recently reported crystal structure of the full-length MMP-2 preform (Morgunova, E. et al., Science 284, 1667-1670 (1999)), along with previous biochemical studies exploring domain properties (Murphy, G. et al., Biochem. J. 283, 637-641 (1992); Fridman, R. et al., J. Biol. Chem. 267, 15398-15405 (1992); Nguyen, Q. et al., Biochemistry 33, 2089-2095 (1994); Ye, Q. Z., et al., Biochemistry 34, 4702-4708 (1995)), predicted the loss of all three MMP-2 functional domains: the Zn²⁺ binding site catalytic domain (which is present in all prokaryotic and eukaryotic metalloproteinases), the three fibronectin type II-like domains, and the hemopexin domain.

In Family 1, sequence analysis revealed that the affected individuals were homoallelic for the same missense mutation, a G to A in codon 101 of exon 2 which predicted the replacement of an arginine by a histidine (R101H). Of particular significance, this mutation occurred within the prodomain, a region highly conserved across species and other members of the MMP gene family and which is involved in the autoproteolytic activation of MMP-2 (Van Wart, H. E. & Birkedal-Hansen, H., Proc. Natl. Acad. Sci. USA 87, 5578-5582 (1990)). Molecular modeling from the X-ray crystallographic coordinates (1ICK7; Morgunova, E. et al., Science 284, 1667-1670 (1999)) suggested a potential mechanism for the mutation's pathogenicity. The mutated arginine is adjacent to the cysteine which forms the activation critical “cysteine switch” mechanism (Van Wart, H. E. & Birkedal-Hansen, H., Proc. Natl. Acad. Sci. USA 87, 5578-5582 (1990)). It has been suggested that this cysteine interacts with the catalytic domain zinc ion and hence regulates the conversion (“switch”) from non-catalytic to catalytic states. Replacement of the adjacent arginine with a histidine residue disrupted two potential salt bridges with Asp106 which in turn could result in destabilization of this “cysteine switch”. This nucleotide substitution was not present in 100 chromosomes from 50 unaffected, unrelated Saudi control individuals and it segregated appropriately within the family, such that the unaffected parents were heteroallelic for the mutation while unaffected siblings were either heteroallelic or had the wild type sequence.

While linkage and haplotype analyses demonstrated that the disease locus in Family 2 mapped to this finite region and argued against locus heterogeneity or a phenocopy, no mutations were detected in the thirteen MMP-2 exonic sequences. Therefore, an undetected homoallelic mutation is presumably in the promoter or intronic regions resulting in absent MMP-2 activity (FIG. 2A). Interestingly, the homoallelic polymorphism was detected in affected individuals in codon 210 of exon 4. This G to T transversion resulted in the replacement of an aspartate with a tyrosine residue (D210Y). However, analysis of 50 unrelated and unaffected members of this particular Saudi tribe revealed the mutation to be a relatively common polymorphism. This SNP may prove useful in future MMP-2 association studies.

These findings identify the first inherited matrix metalloproteinase deficiency to cause a human disease. The most striking clinical findings in the three Saudi families included carpal and tarsal osteolysis, osteoporosis, cortical thinning, interphalangeal erosions, flexion contractures of the large joints, nodular fibrous palmar and plantar pads, and dysmorphic facies (Al-Mayouf, et al., Am. J. Med. Genet. 93, 5-10 (2000); Al Aqeel, A. et al, Am. J. Med. Genet. 93, 11-18 (2000)). Additionally, all affected individuals were significantly growth restricted; less than 3% for height, weight, and head circumference.

The unexpected and counterintuitive discovery, that the deficient activity of this well-characterized gelatinase/collagenase results in an inherited osteolytic and arthritic disorder, provides increased understanding of the in vivo function of MMP-2. Presumably, MMP-2 is critical for bone and extracellular matrix solubilization. Therefore, it would have been expected that lack of MMP-2 activity would cause an osteopetrotic phenotype. As shown herein, MMP-2 deficiency and the resultant extracellular matrix breakdown defect can result in an imbalance between bone synthesis and resorption and result in an overall “osteolytic” phenotype.

### Example 2

### Confirmation of MMP-2 Deficiency by ELISA

### Materials and Methods

ELISA. Patient serum and serum-free conditioned media from cultured fibroblasts, prepared as described in Example 1 were collected and assayed using commercially available kits for MMP-2, MT1-MMP, and TIMP-2 (Amersham Life Science). The manufacturer's protocols for these one-step sandwich ELISAs were followed.

### Results

ELISA for MMP-2 specific detection was performed using serum samples from a number of the Saudi individuals (data not shown). The results corroborated the above biochemical and molecular genetic evidence presented above in Example 1. Moreover, the results confirm that the mutant MMP-2 proteins are stable, and can therefore be biochemically analyzed. Both missense proteins were expressed, albeit in markedly diminished amounts, in serum from affected individuals. Parents of each affected child, obligate heterozygotes, produced approximately half of the normal amounts of MMP-2. In addition, a homozygous wild-type unaffected sibling produced normal amounts. By contrast, the MMP-2 truncation mutant was not detectable in serum.

These results, which clearly demonstrate that MONA-causing mutations are expressed and stable, but not enzymatically active, provide justification for structure-function studies.

### Example 3

### Additional MMP-2 Mutations Associated with MONA Syndrome

As already stated, the multicentric osteolyses are a family of inherited disorders which share the major phenotype of carpal, tarsal, and interphalangeal destruction similar to that seen in the MONA syndrome. The results presented herein have now identified 20 additional unrelated families with MONA-like syndromes. For the purpose of clinical distinction, the diagnoses provided by the referring physicians were maintained. Clinical histories and blood samples were provided by referring physicians. Seven patient samples were analyzed using a combination of zymography (when serum samples were available) and MMP-2 mutation analysis.

This resulted in the identification of two additional unrelated families with MONA (neither of Arab origin) and characterized two novel MMP-2 mutations. In patient M1, a homozygous G→A nonsense mutation in exon 7 was detected. This mutation resulted in the replacement of a tryptophan residue by a stop codon (W387X), and predicted the loss of all three MMP-2 functional domains.

Both patients had no evidence of MMP-2 activity in either serum ELISA or fibroblast biochemical assays. In patient M2, also the product of a consanguineous mating, zymography of serum revealed an absence of detectable MMP-2 activity. A homozygous G→A missense mutation in exon 8 was identified which resulted in an aspartate to arginine substitution (D437N).

### Example 4

### Molecular Structure-function Relationship of MONA-Causing MMP-2 Mutations Western Immunoblotting

To confirm that there can be a lack of correlation between MMP-2 activity and amount of MMP-2 protein, suggesting the existence of different pathways of MMP-2 deficiency, mutants can be generated and evaluated by immunoblotting and biochemical assays.

### Materials and Methods

Immunoblotting. For Western analysis of patient samples, fibroblast cell lysates, cell membrane extracts, and conditioned media samples are run on 7.5 to 10% polyacrylamide gels under denaturing/reducing conditions and electrotransferred to PVDF membranes using a semi-dry blotting system. The membrane is then blocked with 5% dry milk in TBS-Tween (pH 7.6) at room temperature and washed with 0.05% TBS-Tween/10% blocker. The membrane is next incubated overnight at 4° C. with the appropriate anti-human MMP, membrane MMP and TIMP mouse monoclonal antibodies (Chemicon; Santa Cruz), washed with TBS-Tween and incubated at room temperature with horseradish peroxidase-conjugated goat antimouse IgG antibody. Blots are developed using an ECL Western blotting detection system (AmershamPharmacia Biotech, Piscataway).

Generation and expression of MMP-2 missense mutations. Cloning, expression, DNA sequence verification, and activity assay of the full-length, 72 kD human MMP-2 wild-type with both a HSV epitope and His tag fused on the carboxy terminus, was previously accomplished (Aqeel et al., Am J Med Genetics 2000; 93:11-18). This construct will serve as a template for the expression of the mutations and polymorphisms already identified and all novel mutations. Additionally, site-directed mutagenesis, using mutation-specific oligonucleotides, as previously described (Glucksman, M. J. and J. L. Roberts, Methods in Neuroscience, 1995. 23: p296-316; Martignetti, J. A. and J. Brosius, Mol Cell Biol, 1995. 15(3): pl642-50; Cummins, P. M., et al., J Biol Chem, 1999. 274(23): p16003-9) can be performed using the Stratagene Chameleon double-strand mutagenesis kit (Stratagene).

Baculovirus protein expression. Both wild-type MMP-2 and individual mutations are produced and purified from the baculovirus insect cell expression system. Sf9 cells are grown in Sf-900 II media supplemented with 5% fetal bovine serum (Invitrogen) in T75 flasks. Expression seeding is performed at 0.25-0.5 million cells/ml to reach a density of 4 million cells/day. Cells are infected at a density of 1 million cells/day for 3 days with a multiplicity of infection of 5, in serum-free medium (a time course is performed and optimized for wild-type MMP-2 protein production). Cells are centrifuged at 500×gravity for 10 min, and the supernatant further centrifuged at 20,000×g for 20 min at 4° C. The conditioned media of insect cells is then rapidly purified by two chromatographic steps. First, immobilized metal ion affinity chromatography on Co⁺² Talon resin (Clontech) is used to purify the His tagged protein: the column is equilibrated in 100 mM NaH₂PO₄, 10 mM Tris-HCl, 50 mM NaCl (pH8.0) and then washed extensively and bound protein is eluted with wash buffer and 80 mM EDTA: After concentration and washing by ultrafiltration, the sample, in 25 mM Tris-HCl, 20 mM NaCl, 1 mM CaCl₂, 0.01 mM ZnCl₂, (pH8.0), is chromatographed by FPLC on a Mono Q (AP Biotech) anion exchange column with a 25-125 mM NaCl gradient as a “polishing step” to remove minor autoactivation products which can be present during conventional purification schemes. The recombinant MMP-2 protein will be eluted as a single peak at 55-65 mM NaCl.

Assessment of purification to homogeneity and proper folding of expressed proteins is by native polyacrylamide gel electrophoresis. The physical and kinetic properties, including kcat and Kₘ, of these normal and mutant proteins are determined (Cimmins, P. M., et al., J Biol Chem, 1999. 274(23): p16003-9; Tullai, J. W., et al., J Biol Chem, 2000. 275(47): p36514-22).

Kinetic analysis: quantitative fluorescent assays. In addition to zymography to assay activity, quantitative fluorescent assays are performed on patient fibroblast conditioned media and, if necessary, serum samples using appropriate substrates to determine the kinetics of the wild-type and mutant proteins. The amino ends of these inhibitor peptides contain a fluorescent 7-methoxy-coumarin-4-acetyl (MCA) moiety and the 2,4-dinitrophenyl (DNP) quenching groups are on the carboxy terminus. Upon peptide cleavage, fluorescence increases 50-fold (Juliano, L., et al., Biochem Biophys Res Commun, 1990. 173(2): p647-52)

MMP-2 activity is determined by using 50 mM (MCA-Pro-Leu-Ala-Nva-Dpa-Ala-Arg-NH2) (Murphy, G., et al., J Biol Chem, 1994. 269(9): p6632-6), and (MCA-Pro-Leu-Ala-Leu-Trp-Ala-Arg-dnp) (a modification of Netzell-Arnett, et al., Biochemistry, 1993. 32(25): p6427-32). For MMP-2 measurements, the substrate is equilibrated to 37° C. and incubated with 100 ml of sample. After incubation the reaction is terminated with 1 mL of 1.5M acetic acid. The fluorescent intensity is determined using a Perkin Elmer LS50 Spectrophotometer. Fluorescence at excitation and emission wavelengths of 325 nm and 393 nm and excitation and emission slit widths of 10 nm and 20 nm, respectively, are measured. Standard curves are prepared with recombinant human fibroblast MMP-2. Other MMPs may be similarly assayed using the appropriate substrates. For example, general MMP activity is assessed using DABCYL-GABA-Pro-Gln-Gly-Leu-Glu (EDANS)-Ala-Lys-NH₂ as substrate. For these experiments, kinetics are compared between the MMP-2 mutants and wild-type MMP-2. Data are collected during the initial zero-order kinetics of the reaction during which <10% of the substrate is consumed. Determinations of the Kₘ and Vₘₐₓ are quantitated and compared through the use of the ENZFITTER program (BIOSOFT, UK).

Mon ocyte chemoattractant protein -3 (MCP-3). The chemokine MCP-3 (McQuibban, G. A., et al., Science, 2000. 289(5482): p1202-6) is assayed as an MMP-2 substrate by both the expressed wild-type (control) and mutant MMP-2 proteins. The cleavage product, and its ability to act as an antagonist that attenuates the inflammatory response, is explored for the MMP-2 mutations. A highly sensitive quenched fluorescence substrate is synthesized to assay MMP-2 activity towards MCP-3 as a substrate. Specifically, MCA-Gln-Pro-Val-Gly-Ile-Asn-Thr-Ser-DNP is utilized to ascertain kinetic parameters of expressed MMP-2 missense mutations constructs and compared to wild-type controls (Cummins, P. M., et al., J Biol Chem, 1999. 274(23): p16003-9; Juliano, L., et al., Biochem Biophys Res Commun, 1990.173(2): p647-52; Shrimpton, C. N., et al., J Biol Chem, 1997. 272(28): pl7395-9).

As an adjunct to studies of enzyme kinetics with these sensitive synthetic fluorimetric substrates, cleavage of the native protein MCP-3 substrate by wild-type and mutant MMP-2 expressed protein is examined. Utilizing 4-20% SDS-PAGE Tricine gels, both the uncleaved recombinant MCP-3 and its substrate products can be identified (McQuibban, G. A., et al., Science, 2000. 289(5482): p. 1202-6). Fibroblasts from unaffecteds (as control) and affecteds are examined.

To examine other potential physiological substrates, type I and IV collagen degradation assays are used (Brooks, P. C., et al., Cell, 1996. 85(5): p683-93).

To avoid potential biases introduced during protein construction and expression, the potential differences in expression levels, structural integrity, and degradation by endogenous proteases between mutant proteins are examined. First, the yields of all mutations and wild-type constructs are compared. Second, to assure proper folding, mutant proteins are subjected to native PAGE gels under non-reducing conditions and compared to wild-type protein. Potential aberrations in protein folding are also further subjected to analysis by circular dichroism, where no gross structural perturbations should be observed if there is proper folding (as in Cummins, P. M., et al., J Biol Chem, 1999. 274(23): p16003-9). Finally, should protein stability be an issue, a folding assessment, or change in packing, based on thermodynamic and kinetic measurements also is studied by fluorescence measurements.

Conformational changes and stability studies: Circular Dichroism (CD). CD is a sensitive method for detecting net changes (<2%) in secondary structure (Johnson, W. C., Protein Secondary Structure and Circular Dichroism. 1990). Screening of expressed mutant MMP-2 proteins proceeds with enzyme assays and kinetics, followed by CD analysis to examine whether there are any alterations in the net conformation as compared to wild-type. Since only 50-100 μg of purified MMP-2 proteins are needed, a 250 ml culture of insect cells (yielding typically 150 μg of purified protein) is sufficient to provide all samples required for enzyme and kinetic assays and CD analysis. Spectra are collected with an AVIV 60DS spectropolarimeter. Data are collected in the wavelength range of 300 nm-185 nm at 0.2 nm intervals, as previously described (Cummins, P. M., et al., J Biol Chem, 1999. 274(23): p16003-9).

Denaturation Curves by Fluorescence. Protein stability assessment, or change in packing, based on thermodynamic and kinetic measurements also may be studied. Experiments correlating the effect of site-directed mutants on stability of MMP-2 are conducted by monitoring the unfolding of the enzyme as a function of urea concentration (Matouschek, A. and A. R. Fersht, Methods Enzymol, 1991. 202: p82-112; Matouschek, A., et al., Nature, 1990. 346(6283): p440-5). Measurements are taken using a Perkin Elmer LS50B Luminescent Spectrometer equipped with a thermostatically controllable 3 mm cuvette and a rapid mixing head, to insure complete mixing of the solutions before readings are taken. Unfolding is initiated by diluting a 0.25 mg/ml solution of MMP-2 ten-fold in 50 mM HEPES (pH 7.2) containing urea, to a final volume of 0.8 ml. Data are collected in duplicate for wild-type and mutants and the slopes calculated. Specifically, the intrinsic fluorescence at the excitation wavelength of 290 nm and an emission wavelength of 315 nm, and data plotted as log Ku (rate constant for unfolding) versus urea concentration. Stability is measured as a function of ΔΔG (kcal/mol). There are many aromatic residues (Phe and Tyr) near sites chosen for mutation that may act as reporters. A mutant with lower stability is reflected in a higher rate of unfolding.

MT1-MMP and TIMP-2 binding assays. As a first step, the interaction of MMP-2 and soluble MT1-MMP (Valtanen, H., et al., Protein Expr Purif, 2000. 19(1): 66-73; Jo, Y., et al., Biochem J, 2000. 345 Pt 3: 511-9) is explored. Using this methodology, baculovirus expressed and activated soluble MT1-MMP is incubated with pro-MMP-2 mutants alone or in combination with TIMP-2. The degree of pro-MMP-2 activation is then monitored by Western, zymography and fluorescent substrates.

To examine the formation of the MT1-MMP-TIMP-2-pro-MMP-2 ternary complex, a crude membrane fraction of fibrosarcoma cells expressing MT1-MMP is incubated with TIMP-2. Excess TIMP-2 is washed out with buffer by centrifugation and the membrane fraction is incubated with a known quantity of mutant pro-MMP-2. Membranes are washed to remove unbound enzyme and the wash analysed by Western blotting. Upon formation of a ternary complex, the remaining crude membrane fraction containing the complex is incubated with free MT1-MMP (Jo, Y., et al., Biochem J, 2000. 345 Pt 3: 511-9).

The ability of MMP-2 missense mutants to bind TIMP-2 is determined by running enzyme:inhibitor mixtures over a gelatin-agarose column followed by analysis of eluted fractions by gel electrophoresis (Olson, M. W., et al., J Biol Chem, 1997. 272(47): 29975-83). Briefly, preparations of expressed pro-MMP-2 mutants (200 pmol) are combined with recombinant TIMP-2 (600 pmol; Chemicon), in 50 mM Tris HCl (pH 7.5), 150 mM NaCl, 5 mM CaCl₂, 0.01% Brij-35 (final volume of 0.1 mL) and incubated for 40 min at 25° C. These mixtures are applied to a gelatin-agarose column, washed with incubation buffer and then washed with buffer supplemented with 10% DMSO. Fractions are collected for all washes and analyzed by non-denaturing gel electrophoresis. Stoichiometry of pro-MMP-2:TIMP-2 complexes are determined by densitometry using known amounts of standard proteins (Olson, M. W., et al., J Biol Chem, 1997. 272(47): 29975-83). In the event of binding of TIMP-2 by mutants, binding affinities are determined using the fluorimetric enzyme activity assay with the peptide substrate MCA-Pro-Leu-Ala-Nva-Dpa-Ala-Arg-NH₂, as described above. Kinetic constants are determined in the presence and absence of TIMP-2 for each mutant (Murphy, G., et al, J Biol Chem, 1994. 269(9): 6632-6).

Additional and more directed studies of potential interactions may be pursued depending on the specific mutation. For example, mutations that could occur in either the gelatin binding site (Murphy, G., et al., J Biol Chem, 1994. 269(9): 6632-6; Tordai, H. and L. Patthy, Eur J Biochem, 1999. 259(1-2): 513-8), TIMP-2 binding site (Overall, C. M., et al., J Biol Chem, 1999. 274(7): 4421-9), or in the PEX domain (Brooks, P. C., et al., Cell, 1996. 85(5): 683-93), the MMP-2 C -terminus region which interacts with integrin ayp3 and is thus important in localizing active MMP-2 on cell surfaces (Brooks, P. C., et al., Cell, 1996. 85(5): 683-93; Silletti, S., et al., Proc Natl Acad Sci U S A, 2001. 98(1): 119-24). For mutations in the C-terminus, a solid phase integrin-MMP-2 binding assay, as described by Siletti et al Proc Natl Acad Sci U S A, 2001. 98(1): 119-24, is used to examine the effects of MONA-causing MMP-2 mutations on integrin binding.

Molecular Modeling. For structural analysis, mutations will be mapped onto the amino acid sequence of the matrix metalloprotease and then identified in the X-ray diffraction-derived structure (Morgunova, E., et al., Science, 1999. 284(5420): 1667-70) in the Protein Data Bank coordinate file (1CK7) operated by the Research Collaboratory for Structural Bioinfornatics. The Insight II or QUANTA software suites (Molecular Simulations Inc) serve as the graphical server interface and are used for rendering. Energy minimization studies are performed with the newly released version of the Crystallography and NMR system (Brunger, A. T., et al., Acta Crystallogr D Biol Crystallogr, 1998. 54(Pt 5): 905-21). Data refinement includes simulated annealing, structural dynamics, energy minimization, and modeling based on electrostatic and hydrogen bonding criteria.

For TIMP-2/MT1-MMP interaction modeling, molecular properties such as mechanics, distance geometry, and docking predictions are examined with the individual structures determined by X-ray crystallography. A pocket surface is constructed with known structures and building electron density into generated atomic coordinates from dynamically connected three dimensional fragments of the solved metalloprotease structures for further surface calculations. Differences in bond lengths, angles, and other coordinates is calculated as well as root-mean-square deviations between the different crystallographically solved structures.

### Example 5

### Cellular Basis of MONA Pathology/MMP-2 Deficiency

Having identified that MMP-2 deficiency results in skeletal, joint, and wound healing abnormalities, physiologic processes dependent upon extracellular matrix breakdown, the collagenolytic ability of MMP-2 deficient human and mouse fibroblasts was examined. MONA skin fibroblast cell lines already have been established from two unrelated families and unaffected family member controls. Fibroblast cell lines from the hypomorphic mice and their heterozygous and normal control litter mates also have been established.

Analysis of type I collagen dissolution. The importance of this model is underscored by the recent and accumulating evidence that MMP-2 is critical for type I collagen remodeling (Haas, T. L., et al., Am. J. Physiol. Heart Circ. Physiol, 2000. 279(4): H1540-7). The availability of families and biologic specimens has already allowed for the examination of a role for MMP-2 in degradation of type I collagen. Cultured skin fibroblasts obtained from an MMP-2 deficient patient (Saudi family #3-truncation mutation Y244X) were seeded in contact with a reconstituted film of type I collagen fibrils. The homozygous MMP-2 deficient fibroblasts were examined for differences in their ability to degrade type I collagen when compared to normal controls. This was tested in a basal state and following induction of MMP expression by IL-1β and phorbolester. Briefly, 24-well plates are coated with a 1-2 μm film of reconstituted rat tail tendon type I collagen fibrils, and a pellet of 37,500 cells in 25 μl growth medium seeded into the center of each well. Fibroblasts were allowed to attach for 6 hr. and washed (Bell, E., et al., Proc. Natl. Acad. Sci. U.S.A., 1979. 76(3): 1274-8).

While markedly decreased, MMP-2 deficient fibroblasts are able to degrade type I collagen when stimulated TPA (phorbolester) and IL-1β (data not shown). This indicates that, while important, MMP-2 is not critical for type I collagen remodeling, as has been previously suggested.

Proliferation, matrix synthesis and degradation in skin fibroblast populated collagen lattices (FPLC). Patient and MMP-2 deficient mouse function can be further examined by culturing cells within three dimensional collagen lattices (Bell, E., et al., Proc. Natl. Acad. Sci. U.S.A., 1979. 76(3): 1274-8). Cultured dermal fibroblasts incorporated into type I collagen lattices share certain characteristics with fibroblasts within dermis and have been used as an in vitro model of wound contraction. Fibroblast morphology and organization within the lattice and lattice contraction are studied over time. Dermal fibroblast lines for mice, patients and controls have already been established. These lines are maintained in serum free media with insulin transferrin selenium media supplement in order to maintain cells during experiments. Cells are cultured within and on top of collagen lattices as described previously using collagen isolated from rat tail tendon (Bell, E., et al., Proc. Natl. Acad. Sci. U.S.A., 1979. 76(3): 1274-8; Ehrlich, H. P., et al., J. Cell. Physiol, 2000. 184(1): 86-92; Ehrlich et al., J. Cell. Physiol, 2000. 185(3): 432-9; Lee, A. Y., et al., Proc. Natl. Acad. Sci. U.S.A., 1997. 94(9): 4424-9). Lattice diameter is measured every hour for the first seven hours and everyday afterwards using a stereomicroscope, to measure the degree of contraction. Cellular morphology and organization is studied by fixing and processing gels using paraffin embedding procedures. Sections are stained with hematoxylin and eosin prior to microscopic examination.

In vivo wound healing in MMP-2 hypomorphs and knockouts. Numerous studies have suggested the importance of MMPs in acute and chronic wound healing (Agren, M. S., Br. J. Dermatol, 1994. 131(5): 634-40; Buckley-Sturrock, A., et al., J. Cell. Physiol., 1989. 138(1): 70-8; Pilcher, B. K. et al, Ann N.Y. Acad. Sci.,1999. 878: 12-24) The dermal abnormalities in MONA, namely impaired wound healing leading to the development of hypertrophic scarring and the development of fibrocollagenous pads, thus represent an important model for dissecting the role of MMP-2 in tissue repair. A study of keloids and hypertrohpic scars suggested that MMP-2 overexpression is associated with the development of these abnormally healed skin wounds (Neely, A. N., et al., Wound Repair Regen, 1999. 7(3): 166-171.) The process of wound healing is dependent on leukocyte recruitment, keratinocyte proliferation and migration, and angiogenesis. Thus, an in vivo model provides a singular opportunity to examine the multiple components of this system.

To examine the effect of MMP-2 deficiency on wound healing, full-thickness punch biopsies (epidermis and dermis) are performed in wild-type, heterozygous, and homozygous MMP-2 hypomorphs and knockouts. Wound-healing rates are calculated based on the percentage of open wound areas with time following biopsy. Wound beds and surrounding margins are collected at multiple time points postinjury and histologically examined for degree of epithelialization, collagen deposition, density of infiltrating inflammatory cells (neutrophils and monocytes/macrophage, degree of capillary infiltration (neovascularization), and wound contracture.

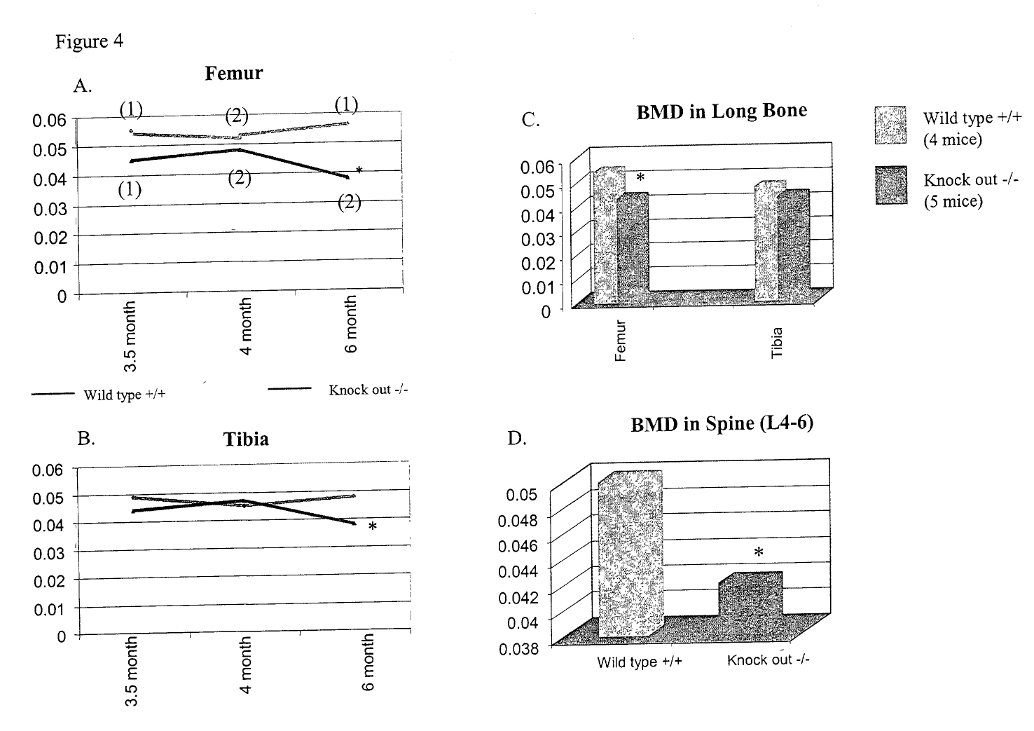

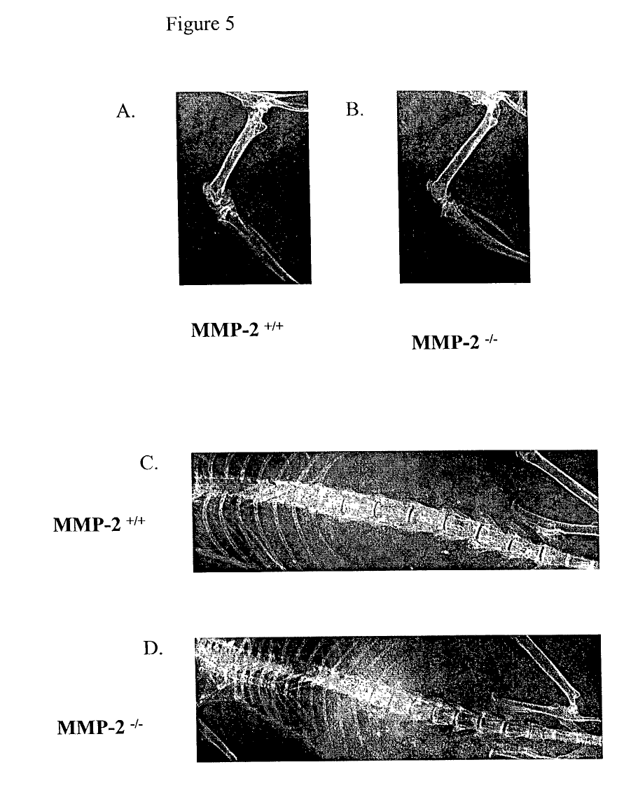

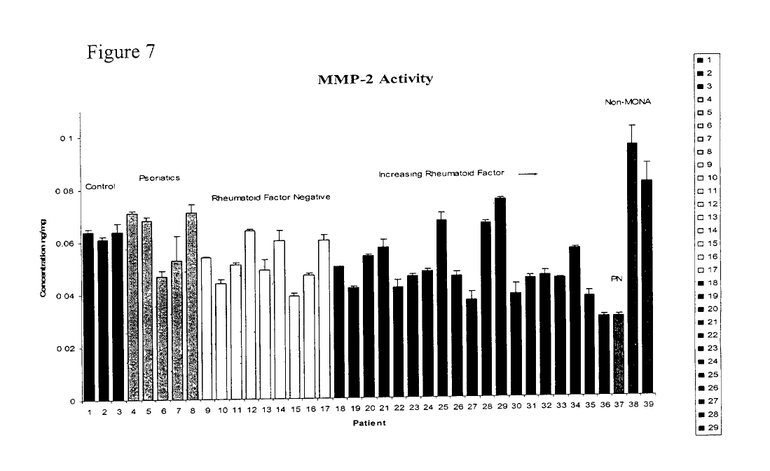

Bone density in MMP-2 deficient Mice. The original MMP-2 knockout mice were generated and described by Itoh and colleagues (Itoh T et al., J Biol Chem. 272:22389-92; 1997) and were directly obtained from Dr. T. Itoh, Kyoto University. These mice were described as being overtly normal except that they were approximately 15% smaller than control littermates (Itoh T et al J Biol Chem. 272:22389-92; 1997). This mild, but obvious, phenotype—possibly secondary to a skeletal defect—had not been previously investigated. Therefore, we performed whole body X-ray imaging of these animals and control littermates (FIG. 4A-D). X-rays suggested a time-dependent loss of bone mineral density in homozygous MMP-2 deficient mice (FIG. 4B and 4D, and FIG. 7) compared with wild-type controls (FIG. 4A and 4C, and FIG. 5). Thus, DEXA (dual energy X-ray absorptometry) studies were performed using age and litter-matched mice. In addition, marked bone density losses were present in femurs and spine from knockout mice when compared to control littermates and this loss occurred in a time-dependent manner (FIG. 5A-D). Therefore, we believe these mice represent an important animal model for studying MMP-2 deficiency.

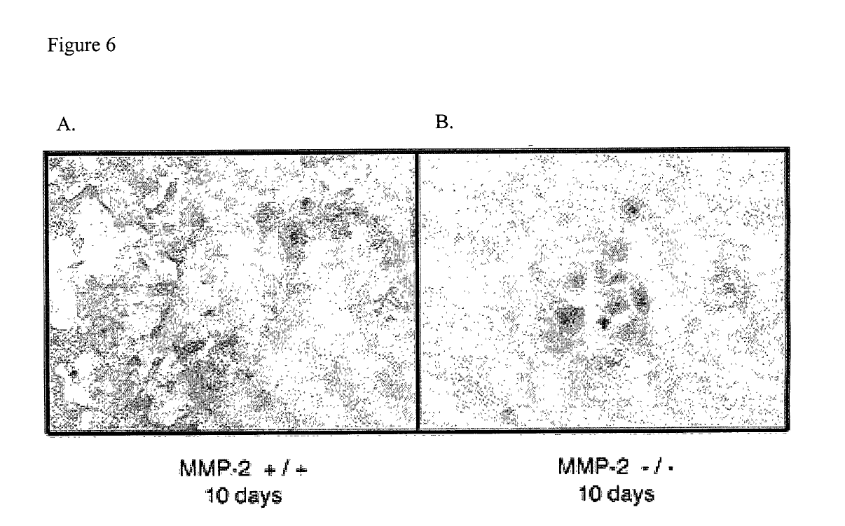

MMP-2 deficiency and osteoblast formation. Experiments examining osteoblastic potential of MMP-2 deficient mouse bone marrow stromal cells have identified significant differences in colony forming efficiency. Mouse bone marrow cells were isolated from paired homozygous MMP-2 deficient and wild-type mice and plated in the presence of ascorbate and washed after 36 hours to remove non-adherent cells. Only wild-type cells, and no MMP-2 deficient cells were detected with this washing protocol. The wild-type cells formed colonies (FIG. 6A) and large mineralized clusters of alkaline phosphatase (AP) staining osteoblasts (FIG. 6B). Cultures were repeated with washing performed at day 5. At day 10, while wild-type cells formed large and numerous colonies of AP positive fibroblasts, MMP-2 deficient colonies were sparse and low in cell number. This data is suggestive of a possible defect in the CFU-F efficiency of MMP-2 deficient bone marrow.

MMP-2 knockouts express low-level amounts of active MMP-2. In an attempt to investigate the apparent discrepancy between human and mouse deficiency phenotypes, we re-examined the MMP-2 knockouts provided us by Itoh et al (Itoh et al., J Biol Chem 1997; 72:22389-92) by first attempting to confirm their “knockout” status. Having confirmed the genotype, serum zymography was performed and revealed low-levels of active MMP-2 in genotypically confirmed “knockout mice”. As a control, littermate heterozygotes were shown to possess half-normal levels when compared to their littermate normals (data not shown). We estimate the MMP-2 level to be approximately ˜3% of wild-type control.

Using an MMP-2-specific antibody based activity system (Amersham), the “hypomorphic” mice were also shown to produce ˜5% active MMP-2. Serum samples were collected from wild-type, heterozygous and homozygous mice and incubated in a microtitre plate coated with a specific MMP-2 antibody, which does not cross-react with other MMPs. After washing, bound MMP-2 was APMA (p-aminophenylmercuric acetate) activated and incubated with a MMP-2-activated detection enzyme and chromogenic peptide substrate.

Further evidence that MMP-2 RNA was being transcribed was provided by RT-PCR. Liver RNA was extracted from knockout and control wild-type mice (data not shown). Two independent pairs of MMP-2 specific primers directed against the mouse cDNA sequence, expected to produce products of 890 bp and 2 kb, were tested. The highly homologous human sequence (>95% identity between mouse, rat, and human mRNA se ward: 5′ GTG GAT GAT GCT TTT GCT GGG G 3′; reverse 5′ CAC AGA GTG AGG AGG GGA ACC 3′) were designed, prior to the release of the mouse DNA sequence NM_{—}008610, against sequence corresponding to the homologous human exons 2 and 13. As predicted from the cDNA sequence, a 2 kb product was amplified and confirmed to be mouse MMP-2 by direct sequence analysis. Amplification of contaminating genomic DNA, and hence introns, would have resulted in a significantly larger product.

Together, these results strongly suggest that the originally generated MMP-2 deficient mice (Itoh et al., J Biol Chem 1997; 272:22389-92) produce MMP-2 message and active protein, and are thus “hypomorphs”.

### Example 6

### Generation and Characterization of MMP-2 Knockout Mice

MMP-2 hypomorphs and knockout provide a useful model for investigating the function of MMP-2. The developmental consequences of loss-of-function mutations in paired combinations, especially MMP-2 and MT1-MMP, which are individually known to play a role in the formation of bone, help to elucidate their functional overlap and cascade of activity.

Define the skeletal cell-type and developmental expression pattern of MMP-2. To determine the tissue distribution of MMP-2, RNA in situ hybridization analysis will be performed. These studies are performed on fixed and embedded sections. ³⁵S RNA-MMP-2 specific sense and antisense probes are synthesized from subcloned DNA fragments using vector derived promoters. The specificity of these 3′ end probes are established by probing total genomic DNA by Southern blotting. Following hybridization and washing, the sections are air dried and exposed overnight to film to determine signal strength. Autoradiography is performed by dipping the slides in a 1:3 ratio of water:Kodak NBT2 emulsion, air drying and exposing for 3-7 days. Developing in Kodak D19 and hematoxylin counter staining then follows. Folowing fixation, embryos are dehydrated in graded methanols and stored at −20° C. Embryos are then rehydrated and briefly digested with proteinase K. Digoxygenin (DIG) labeled RNA probes are prepared and hybridized at 65° C. overnight. Following washing in PBS and Tween, embryos are incubated in a 1:2000 dilution AP-anti-DIG antibody followed by washing and staining in Purple Precipitating Reagent at 4° C. Stained embryos are photographed as whole mounts, then dehydrated with ethanol, washed with acetone, and incubated ovenight in a 1:1 mixture of acetone and araldite. Embryos are then transferred into a mold with new araldite and incubated for 16 hours at 80° C. for polymerization. Subsequently, sections are cut, mounted in araldite and photographed under Nomarski optics.

Analysis of MMP-2 hypomorphic mice. These experiments were carried out using a breeding colony of MMP-2 hypomorphic mice. These mice were originally generated and described by Itoh and colleagues (Itoh, T., et al., J. Biol. Chem., 1997. 272(36): 22389-92). Based on the results identifying MMP-2 mutations as the cause of MONA, it is hypothesized that the mouse growth defect, homozygotes are approximately 15% smaller than control littermates, is secondary to skeletal defects. Thus, detailed histologic and ultrastructural examination of the axial, appendicular and craniofacial skeletons and synovial tissue of MMP-2 hypomorphs are conducted. Briefly, radiologic analysis is done by whole-body X-ray imaging of hypomorphs and control littermates. For determination of the rate of longitudinal bone growth, mice of each genotype are injected intrapertoneally with the fluorochrome calcein (10 mg/kg of body weight) 4 or more days before sacrifice. The tibiae of calcein-injected animals are fixed in 2.5% formaldehyde, dehydrated in graded ethanol and embedded in parafin. 10 μM frontal sections are cut and viewed using fluorescent microscopy. Distances between the zone of vascular invasion within the growth plate and the proximal end of the calcein label in the metaphysis are measured. The height of the growth plate is determined and the daily growth rate calculated.

For histologic studies, paraffin embedded sections are collected on glass slides, dewaxed and stained with either: hematoxylin and eosin (HE); alcian blue and nuclear fast red (AR), or hematoxylin, fast green, and basic fuschin (HGF) as previously described (Tribioli, C. et al., Development, 1999. 126(24): p. 5699-711). Embryos are be fixed in paraformaldehyde and then dehydrated through ethanol gradients, followed by Americlear and paraffin embedding. AR staining for cartilage is performed on dewaxed and rehydrated sections. Slides are treated with 1% alcian blue 8GX in 3% glacial acetic acid, followed by washing in running water, and then counter stained in nuclear fast red. Sections are dehydrated in graded ethanols and coverslipped. HGF staining for collagen-associated proteoglycans is performed as follows. Rehydrated sections are stained in Weigert's iron hematoxylin solution and rinsed with running water until the blue color fully develops. Sections are then transferred to fast green FCF stain, rinsed briefly in 1% acetic acid, and then stained in 0.1% basic fuschin. Sections are then dehydrated in 95% ethanol and 100% ethanol and coverslipped. Mineralization is assessed by Von Kossa staining.

For staining and visualization of whole mount cartilage and ossified skeletal elements, embryos or neonatal mice are dissected and stained with alizarin red and/or alcian blue. For alcian blue/alizarin red combined staining, the skin and internal organs are removed and the samples fixed overnight in 95% ethanol followed by staining with 0.02% alcian blue in a 4:1 95% ethanol:glacial acetic acid solution. The samples are washed in 95% ethanol and immersed in 2% KOH for several hours. The samples are then stained in alizarin red in 1% KOH, then processed through a graded series of glycerols in ethanol and stored in 100% glycerol. For cartilage staining, embryos are fixed in Bouin's solution overnight, rinsed with water several times, immersed in four changes of 1% ammonia in 70% ethanol for at least one hour each and stained with 0.05% alcian blue in 5% acetic acid. Embryos are rinsed in 5% acetic acid. Specimens are dehydrated through graded series of ethanols, cleared and stored and photographed.

If no skeletal defects are identified, the role of known metabolic growth factors is investigated.

Generate and characterize MMP-2 knockout and MMP-2/MT1-MMP and MMP-2/TIMP-2 double knockout mice. To generate a MMP-2 null mouse, a mouse genomic SV129 λ Fix II bacteriophage library (Stratagene) is screened using the mouse MMP-2 cDNA as a probe; as previously described (Holmbeck, K., et al., Cell, 1999. 99(1): 81-92; Caterina, J., et al, Ann N.Y. Acad. Sci, 1999. 878: 528-30; Hou, W. S., et al., J. Clin. Invest, 1999. 103(5): 731-8). To generate the targeting vector, restriction fragments containing multiple exons from the middle to terminal portion of the gene are sought. If full-length clones are not obtained in this library, a P1 (Research Genetics) library is screened.

The pBS II SK+vector (Stratagene) is used as the targeting vector construct for cloning of the two homologous 5′ and 3′ regions. The “middle” portion of the insert is replaced with a phosphoglycerate kinase promoter-driven HPRT minigene cassette. The targeting vector is completed by addition of an HSV-tk minigene. The construct is linearized, purified, and electroporated into an ES cell line and selected for by treatment with HAT supplement treated growth media and ganciclovir (Roche Laboratories). Surviving clones are expanded and genotyped by PCR to assay for the endogenous and mutant loci and Southern blotted for further characterization of the gene insert. To generate chimeric mice, positive ES cells are injected into 3 day-old blastocysts from C57BL/6 mice and implanted into pseudopregnant C57BL/6×DBA females. Offspring are mated to Black Swiss mice to generate heterozygous animals. These are then interbred to generate homozygous progeny.

The MT1-MMP and TIMP-2 knockouts are provided by Dr. Birkedal-Hansen (National Institute of Dental and Craniofacial Research), who originally generated and characterized these knockouts (Holmbeck, K., et al., Cell, 1999. 99(1): 81-92; Caterina, J., et al., Ann N.Y. Acad. Sci, 1999. 878: 528-30; Zhou et al., Proc. Nat. Acad. Sci. USA, 2000. 97:4052-57; and Caterina J., et al., J. Biol. Chem. 2000. 275:26416-22). All three knockouts, MMP-2, MT1-MMP, and TIMP-2, share the C57BL/6 genetic background and the MT1-MMP, and TIMP-2 knockouts are known to be fertile. Moreover, all three genes are known to be present on unique murine chromosomes: MMP-2 (chromosome 8), MT1-MMP (chromosome 14), and TIMP-2 (chromosome 11). Double knockouts are obtained by intercrossing pairs of heterozygous mice; in this manner, control littermates also are generated. Genotyping of animals is performed by either Southern blot analysis of PCR amplification of DNA obtained from tail biopsies, as previously described (Itoh, T., et al., J. Biol. Chem., 1997. 272(36): 22389-92; Holmbeck, K., et al., Cell, 1999. 99(1): 81-92; Caterina, J., et al., Ann N.Y. Acad. Sci, 1999. 878: 528-30. Northern analysis, Western blotting, and activity assays are used as additional confirmatory tests.

Analysis of these mice will depend on the phenotype. If viable, skeletal analysis will proceed as described above. If however, the double knockout phenotype(s) is(are) lethal, the exact gestational time-points and underlying mechanism of the lethality is pursued. If lethal, one possibility is to generate crosses using the hypomorphic MMP-2 model.

### Example 7

### Definition of the Genotype/Phenotype Correlates of MONA

The overall objective of this Example is to define the genotype/phenotype correlates of MONA. Newly identified osteolysis families are clinically and radiologically characterized.

Clinical characterization of MONA families. Affected indidivuals are examined in the Mount Sinai General Clinical Research Center (GCRC) under an IRB-approved protocol to better define the natural history, clinical variability, and spectrum of MONA manifestations. A complete history is obtained and physical examination performed. Patients are considered affected if they have positive X-ray findings consistent with MONA and MMP-2 deficiency, as assayed by zymography. These X-rays also constituted baseline evaluations for the age-related changes. (Ehrlich, H. P., et al., J. Cell. Physiol, 2000. 185(3): 432-9). Technetium HDP scans are used to detect areas of increased bone turnover and to correlate them with the patient's X-ray findings. Bone densitometry studies using DEXA (dual emission X-ray absorbtometry) are performed on post-pubertal individuals (where calibration values exist) to evaluate possible mechanical alterations in bone formation. Blood and skin biopsy samples are obtained with informed consent from all probands and relevant family members for the purpose of establishing immortalized lymphoblastoid and fibroblast cell lines. All family members identified as carriers are offered genetic counseling.

Biochemical and genetic analysis of MONA families. For affecteds found to have MMP-2 deficiency or gene mutations, further studies are performed as described. Studies in this Example are focused on identifying and characterizing those individuals with MMP-2, MT1-MMP, or TIMP-2 defects.

Zymography and reverse zymography: These qualitative/semi-quantitative assays for MMP-2 activity are performed on serum samples or serum-free conditioned fibroblast. TIMP-2 inhibitory activity is detected by reverse zymography. This is achieved by adding 25 ng/ml gelatinase A (Chemicon), or baculovirus produced MMP-2 to gelatin gel prior to polymerization. Dark zones against a clear background indicate TIMP-2 activity.

Western immunoblotting. For Western analysis of patient samples, fibroblast cell lysates, cell membrane extracts, and conditioned media samples are run on 7.5 to 10% polyacrylamide gels under denaturing/reducing conditions and electrotransferred to PVDF membranes using a semi-dry blotting system. The membrane is blocked with 5% dry milk in TBS-Tween (pH 7.6) at room temperature and washed with 0.05% TBS-Tween/10% blocker. The membrane is then incubated overnight at 4° C. with the appropriate anti-human MMP, membrane MMP and TIMP mouse monoclonal antibodies (Chemicon; Santa Cruz), washed with TBS-Tween and incubated at room temperature with horseradish peroxidase-conjugated goat antimouse IgG antibody. Blots are developed using an ECL Western blotting detection system (AmershamPharmacia Biotech, Piscataway).

ELISA. Patient serum and serum-free conditioned media from cultured fibroblasts, prepared as described above, are collected and assayed using commercially available kits for MMP-2, MTl-MMP, and TIMP-2 (Amersham Life Science). The manufacturer's protocols for these one-step sandwich ELISAs are followed.

DNA sequence mutation detection: DNA sequence analysis. PCR primers are designed to amplify each exon and the respective flanking intron/exon sequences of the MMP-2, MT1-MMP, and TIMP-2 genes from affected individuals and non-affected family members. PCR amplifications are carried out as previously described (Consortium, Nat. Genet, 2000. 26(1): 103-5). All exons have been successfully amplified using the following PCR cycle conditions: initial denaturation at 96° C. for 10 min followed by 30 cycles, each at 96° C. for 30 s, 55° C. for 30 s, and 72° C. for 1 min, and a final extension of 72° C. for 5 min. Data are analyzed using the ABI Sequencing Analysis 3.3 (Perkin Elmer) and Sequencher 3.11 (Gene Codes Corporation) software programs

Denaturing high performance liquid chromatography. As an adjunct to direct DNA sequencing, patient-derived PCR samples are assayed by denaturing high performance liquid chromatography (DHPLC). DHPLC has a far higher mutation detection rate compared to other screening methods such as single stranded conformational polymorphism (SSCP) analysis and denaturing gradient gel electrophoresis (Gross, E., et al., Hum Genet, 1999 105(1-2): 72-8; Oldenburg, J., et al., A. J. Biochem Biophys. Methods, 2001. 47(1-2): 39-51; Roberts, P. S., et al., J. Biochem. Biophys. Methods, 2001. 47(1-2): 33-7). Heteroduplexes and mutant homoduplexes melt at different temperatures compared to wild-type homoduplexes and are detected by U.V. light absorbance. MMP-2, TIMP-2, and MTI-MMP specific denaturing gradient and temperature profiles are titrated. Any variant detected is sequenced and the pathogenicity is assessed. Ultimately the success of the DHPLC system provides not only a lower-cost alternative to DNA sequencing for screening but also provides the basis for MMP SNP-association analyses in a number of arthritic and skeletal disorders. This method is faster and markedly less expensive than DNA sequencing, as no post-PCR manipulation is required and the data analysis is not operator dependent. DNA samples are separated by size and sequence depending on the denaturing gradient and temperature profiles used in their analysis.

To confirm that sequence changes are disease-causing mutations and not polymorphisms, alleles from 200 chromosomes of non-affected control individuals are analyzed. Also, all individuals from each family are sequenced to assure that the mutation segregates appropriately with disease status.

### Example 8

### Biochemical Characterization of MMP-2 in Arthritic Patients

The objective of this example was to determine if there was a correlation the protein levels of MMP-2 in patients who are suffering from arthritic conditions and correlate these levels with MMP-2 biochemical activity.

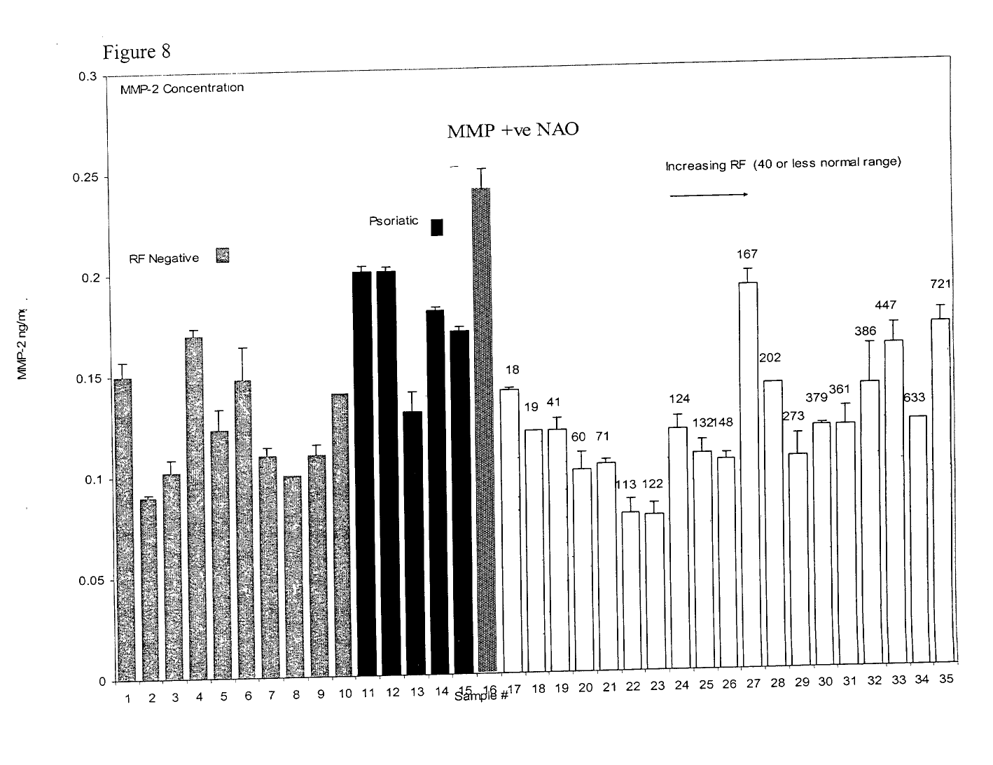

Serum samples from patients suffering from arthritis and other disease conditions were collected and assayed for MMP-2 protein levels using commercially available ELISA kits for MMP-2. The same serum samples were also assayed for MMP-2 activity using commercially available kit (Chemicon) and quantified using a standard MMP-2 provided by Chemicon. The results clearly show that there is a substantial decrease in MMP-2 activity in serum samples from. patient with arthritic conditions compared to samples from non-arthritic patients (FIG. 7). The same samples when assayed for MMP2-protein levels has not shown corresponding reduction in levels of MMP-2 protein (FIG. 8). These results suggest that MMP-2 deficiency is not necessarily caused by decreased amounts of MMP-2 protein, but rather, can also result from decreased activity. Therefore, these result show that MMP-2 activity regulation is mediated by more than one mechanism.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed:

1. A method for the prevention or treatment of a disease mediated by a deficiency of MMP-2 activity in a subject, which method comprises stimulating MMP-2 activity in the subject.

2. The method according to claim 1, which comprises administering to the subject in need of such treatment an effective amount of a substance that stimulates MMP-2 activity, with a pharmaceutically acceptable carrier.

3. The method according to claim 2, wherein the substance that stimulates MMP-2 activity is a MT1-MMP protein or gene therapy vector.

4. The method according to claim 1, which comprises administering to the subject in need of such treatment an effective amount of a vector that encodes an MMP-2 protein, with a pharmaceutically acceptable carrier.

5. The method according to claim 4, wherein the vector is a DNA vector.

6. The method according to claim 1, which comprises administering to the subject in need of such treatment an effective amount of an MMP-2 protein, with a pharmaceutically acceptable carrier.

7. The method according to claim 1, which comprises administering to the subject in need of such treatment an effective amount of a TIMP-2 suppressor.

8. The method according to claim 6, wherein the TIMP-2 suppressor is an anti-TIMP-2 antibody.

9. The method according to claim 1, wherein the disease involves arthritis.

10. The method according to claim 1, wherein the disease involves osteolysis.

11. The method according to claim 1, wherein the disease involves osteopenia or osteoporosis.

12. The method according to claim 1, wherein the disease involves hirsutism.

13. The method according to claim 1, wherein the disease involves abnormal wound healing.

14. The method according to claim 1, wherein a route of administration is topical.

15. The method according to claim 1, wherein the disease results from a mutation in a gene for MMP-2 that results in a defect in expression of MMP-2.

16. The method according to claim 15, which comprises administering to the subject in need of such treatment an effective amount of an MMP-2 protein, with a pharmaceutically acceptable carrier.

17. The method according to claim 15, which comprises administering to the subject in need of such treatment an effective amount of a substance that stimulates MMP-2 activity, with a pharmaceutically acceptable carrier.

18. The method according to claim 15, which comprises administering to the subject in need of such treatment an effective amount of a vector that encodes an MMP-2 protein, with a pharmaceutically acceptable carrier.

19. The method according to claim 15, which comprises administering to the subject in need of such treatment an effective amount of a TIMP-2 suppressor.

20. The method according to claim 1, which comprises stimulating MMP-2 activity in bone marrow cells.

21. A pharmaceutical composition comprising a nucleic acid that encodes an MMP-2 protein, with a pharmaceutically acceptable carrier.

22. A pharmaceutical composition comprising an MMP-2 protein, with a pharmaceutically acceptable carrier.

23. A method for identifying a substance useful in the prevention or treatment of a disease mediated by a deficiency of MMP-2 activity in a subject, which method comprises determining the effect of the substance on a biological activity of MMP-2 protein, wherein a stimulatory effect is indicative of a substance useful in the prevention or treatment of a disease mediated by a deficiency of MMP-2 activity in a subject.

24. The method according to claim 23, which comprises modeling binding of a compound to a site on a structural model of a mutant MMP-2.

25. The method according to claim 24, which comprises modeling binding of a compound to a site on a structural model of MMP-2, wherein a stop codon substitutes for a tyrosine at position 244.

26. The method according to claim 24, wherein the compound was identified in a screen for the ability to stimulate MMP-2 protein activity.

27. The method according to claim 24, wherein the compound is designed using a de novo rational drug design approach.

28. The method according to claim 23, wherein determining the effect of the substance on a biological activity of MMP-2 protein encompasses determining whether the substance has an agonist effect toward binding of MT1-MMP to MMP-2, whereby MMP-2 is activated.

29. The method according to claim 23, wherein determining the effect of the substance on a biological activity of MMP-2 protein encompasses determining whether the substance has an antagonist effect toward binding of TIMP-2 to MMP-2, whereby MMP-2 inhibition by TIMP-2 is blocked.

30. A method for diagnosing a disease mediated by a deficiency of MMP-2 activity in a subject, which method comprises assessing the level of expression of MMP-2 in a biological sample of a test subject and comparing it to the level of expression of MMP-2 in a control sample, wherein a decrease of expression of MMP-2 in the sample of the test subject compared to the control sample is indicative of an extracellular matrix breakdown defect in the test subject.

31. The method according to claim 30, wherein the disease involves arthritis.

32. The method according to claim 30, wherein the disease involves osteolysis.

33. The method according to claim 30, wherein the disease involves osteopenia or osteoporosis.

34. The method according to claim 30, wherein the disease involves, wherein the disease involves hirsutism.

35. The method according to claim 30, wherein the disease involves, abnormal wound healing.

36. The method according to claim 30, wherein the level of expression of MMP-2 is assessed by determining the quantity of MMP-2 protein present in the biological sample.

37. The method according to claim 30, wherein the level of expression of MMP-2 is assessed by assaying the quantity of mRNA which is present in the biological sample and encodes MMP-2.

38. A method for the prevention or treatment of baldness or alopecia in a subject, which method comprises administering to the subject in need of such treatment an effective amount of a substance that inhibits MMP-2 activity, with a pharmaceutically acceptable carrier.

39. A gene encoding a MMP-2, wherein the gene is mutated, which results in a defect in expression of a functional MMP-2.

40. The gene of claim 39, wherein the mutation is selected from the group consisting of an insertion in the gene, a deletion of the gene, a truncation of the gene, a nonsense mutation, a frameshift mutation, a splice-site mutation, and a missense mutation.

41. The gene of claim 39, wherein the mutation is a TCA to TAA nucleotide change in codon 244 of exon 5 resulting in a nonsense codon.

42. A mutant MMP-2 protein which is non-functional.

43. The mutant MMP-2 protein of claim 42 which is truncated and lacks an enzyme active site domain, selected from the group consisting of MMP-2 having a stop codon at position 244.

44. A method for detecting a genetic mutation associated with a bone disease in a mammal comprising detecting a mutation in a gene for MMP-2.

45. The method according to claim 44, wherein the disease is arthritis.

46. The method according to claim 44, wherein the disease is osteolysis.

47. The method according to claim 44, wherein the disease involves osteopenia or osteoporosis.

48. The method according to claim 44, wherein the mutation is a TCA to TAA nucleotide change in codon 244 of exon5.

49. The method according to claim 44, wherein the mutation is selected from the group consisting of an insertion in the gene, a deletion of the gene, a truncation of the gene, a nonsense mutation, a frameshift mutation, a splice-site mutation, and a missense mutation.

50. A method for diagnosing a bone disease comprising detecting a mutation in a gene for MMP-2 that results in a defect in expression of a functional MMP-2.

51. The method according to claim 50, wherein the mutation is selected from the group consisting of an insertion in the gene, a deletion of the gene, a truncation of the gene, a nonsense mutation, a frameshift mutation, a splice-site mutation, and a missense mutation.

52. The method according to claim 50, wherein the bone disease is arthritis.

53. A method for predicting the likelihood of developing bone disease comprising detecting a mutation in a gene for MMP-2 that results in a defect in expression of a functional MMP-2, and determining that there is a likelihood of developing bone disease if the mutation is present.

54. The method according to claim 53, wherein the mutation is selected from the group consisting of an insertion in the gene, a deletion of the gene, a truncation of the gene, a nonsense mutation, a frameshift mutation, a splice-site mutation, and a missense mutation.

55. The method according to claim 54, wherein the mutation is a TCA to TAA nucleotide change in codon 244 of exon 5.

56. A kit for detecting a genetic mutation in a gene for MMP-2 that results in a defect in expression of a functional MMP-2, comprising an oligonucleotide that specifically hybridizes to or adjacent to a site of a mutation of the gene for MMP-2 that results in a defect in expression of a functional MMP-2.

57. The kit according to claim 56, wherein the oligonucleotide is a labelled probe having a sequence corresponding to the sequence of the gene encoding MMP-2 at the site of the mutation, whereby hybridization of the probe is indicative of the presence of the mutation.

58. The kit according to claim 56, wherein the oligonucleotide hybridizes to a first site adjacent to the site of the mutation, further comprising a second oligonucleotide that specifically hybridizes to a second site adjacent to the site of the mutation, wherein the second site is on the opposite strand relative to the first site, and oriented relative to the first site such that both sites flank opposite sides of the site of the mutation, whereby the first and second oligonucleotides serve as primers for PCR amplification of the site of the mutation.

59. The kit according to claim 56, wherein the mutation is a TCA to TAA nucleotide change in codon 244 of exon 5.

60. A method for detecting an intracellular macromolecule associates with MMP-2 comprising:
a) contacting an MMP-2 protein with a candidate macromolecule under conditions that permit association of the MMP-2 protein with a macromolecule; and
b) identifying a macromolecule that associates with the MMP-2 protein.

61. The method according to claim 60, further comprising identifying a mutation in a nucleic acid encoding the macromolecule in a genomic DNA sample from a subject suffering from a bone disease who does not have a defect in expression of a functional MMP-2 protein.

62. The method according to claim 60, wherein the macromolecule is a protein.

63. The method according to claim 60, wherein the macromolecule is a nucleic acid.

64. A method of treating an arthritis in a subject suffering from arthritis, which method comprises administering a functional level of MMP-2 into cells of the subject.

65. A method of screening for a candidate compound that modulates activity of MMP-2, comprising detecting binding of MMP-2 with a compound and isolating the compound.

66. The method according to claim 65, wherein the MMP-2 is a mutant form of MMP-2.

67. The method according to claim 65, wherein the mutant form of MMP-2 has a TCA to TAA nucleotide change in codon 244 of exon 5.

68. A kit for screening for a candidate compound that modulates the activity of MMP-2, comprising an MMP-2 polypeptide and a detector of binding of MMP-2 to a compound.

69. The kit of claim 59, wherein the MMP-2 is a mutant form of MMP-2.

70. The kit of claim 60, wherein the mutant form of MMP-2 has a TCA to TAA nucleotide change in codon 244 of exon 5.

71. A method for diagnosing a bone disease mediated by a deficiency of MMP-2 activity in a subject, wherein the levels of MMP-2 protein are unaltered, wherein the levels of MMP-2 activity is decreased, which method comprises assessing the expression of MMP-2 protein and activity in a biological sample of a test subject and comparing it to the level of MMP-2 protein and activity in a control sample.

72. The method according to claim 71, wherein the disease involves arthritis.

73. The method according to claim 71, wherein the disease involves osteolysis.

74. The method according to claim 71, wherein the disease involves osteopenia or osfeoporosis.

75. The method according to claim 71, wherein the disease involves hirsutism.

76. The method according to claim 71, wherein the disease involves abnormal wound healing.

77. A method for diagnosing a bone disease mediated by a deficiency of MMP-2 activity in a subject, wherein the levels of MMP-2 protein are increased but the levels of MMP-2 activity is decreased, which method comprises assessing the MMP-2 protein and activity in a biological sample of a test subject and comparing it to the level of MMP-2 protein and activity in a control sample.

78. The method according to claim 77, wherein the disease involves arthritis.
